(19) United States
(12) Patent Application Publication
(10) Pub. No.: US 2024/0299532 A1
(43) Pub. Date: Sep. 12, 2024

(54) SUBUNIT VACCINES WITH DINUCLEOTIDE-LOADED HYDROGEL ADJUVANT

(71) Applicant: The Board of Trustees of the Leland Stanford Junior University, Stanford, CA (US)

(72) Inventors: Eric Andrew Appel, Palo Alto, CA (US); Emily C. Gale, Stanford, CA (US); Lingyin Li, Stanford, CA (US); Lauren J. Lahey, Los Altos, CA (US)

(73) Assignee: The Board of Trustees of the Leland Stanford Junior University, Stanford, CA (US)

(21) Appl. No.: 18/549,722

(22) PCT Filed: Mar. 9, 2022
(86) PCT No.: PCT/US2022/019606

§ 371 Date: Sep. 8, 2023

Related U.S. Application Data
(60) Provisional application No. 63/159,416, filed on Mar. 10, 2021.

(51) Int. Cl. A61K 39/215; A61K 39/00; A61M 5/178; A61P 37/04

(52) U.S. Cl. CPC A61K 39/215; A61M 5/178; A61P 37/04; A61K 2039/54; A61K 2039/55505; A61K 2039/55511; A61K 2039/5555; A61K 2039/55561; A61K 2039/55572; A61K 2039/55577; A61K 2039/6087; A61K 2039/6093

(57) ABSTRACT

Provided herein are vaccine delivery systems including a polymer hydrogel non-covalently crossed-linked with a plurality of nanoparticles, a dinucleotide adjuvant encapsulated in the hydrogel, and an antigen encapsulated in the hydrogel. The provided vaccine delivery systems are particularly useful for slowly releasing the antigen and adjuvant within a subject, thereby triggering a more therapeutically effective immune response. Also provided are kits including the disclosed vaccine delivery systems, and methods of using the disclosed materials.

### CROSS-REFERENCE TO RELATED APPLICATIONS

The present application claims priority to U.S. Provisional Application No. 63/159,416, filed Mar. 10, 2021, the full disclosure of which is incorporated by reference in its entirety for all purposes.

### BACKGROUND

Vaccines are among the most effective medical interventions in history. The eradication of smallpox, near eradication of poliomyelitis, and vast decreases in diphtheria, measles, and rubella are testaments to the ability of vaccines to transform disease burden worldwide. It is estimated that vaccines have prevented 103 million cases of disease in the United States since 1924, and saved 2.5 million lives worldwide per year.

Innovations in vaccine design have the potential to improve current vaccines and pave the way for creating new vaccines. Traditional vaccine design is based on using attenuated or inactivated live viruses, which provide cues to the immune system to create an immune memory without causing illness in patients. These whole pathogen vaccines do not allow for targeted immune responses because they contain multiple antigens and innate immune-activating molecules. In contrast, subunit vaccines are composed of a purified antigen (often a protein) from the microorganism and an adjuvant to stimulate the immune system. These vaccines drive highly specific antigen targeting and remove many of the safety challenges associated with using whole microorganisms. Subunit vaccines have become more widely used for infectious diseases, though they have limited ability to produce robust and persistent immune responses for many target diseases.

A failure of subunit vaccines to elicit a sufficiently strong immune response likely arises, in part, from inappropriate temporal control over antigen presentation and adjuvant mediated activation. Natural infections expose the immune system to antigen and inflammatory signals for 1-2 weeks. Conversely, the short-term presentation of subunit vaccines from a single bolus administration persists for only 1-2 days. Recent work demonstrates that the kinetics of antigen presentation to the immune system dramatically influences the adaptive immune response. Previous biomaterial solutions for prolonged vaccine delivery have relied on polymer microparticles whose synthesis typically requires organic solvents that can denature biologic cargo. Further, vaccines and other immunotherapies are typically administered in a saline solution as a series of multiple shots in order to achieve appropriate responses. These are commonly combinations of multiple compounds that can differ greatly in molecular weight and/or chemical makeup, complicating their co-release.

Additionally, although recent clinical successes have demonstrated that immunotherapy affords exceptional potential as a treatment strategy leading to complete eradication of primary tumors and metastases, patients exhibit highly variable responses. Combining multiple approaches engaging both innate and adaptive immune responses may improve response rates, and multi-agent immunotherapies are already in clinical testing. Molecular reagents of these tested immunotherapies include adjuvants, cytokines, and antigens ranging from peptides to antibodies to nucleic acids to lipopolysaccharides. To appropriately activate the immune response, these reagents must often be present in the same place at the same time; however, proper timing is extremely challenging or often impossible when the components differ in chemical nature. Immunostimulatory molecules such as the cyclic dinucleotide cGAMP and the short oligo dinucleotide CpG have shown promise as adjuvants in both prophylactic vaccines and cancer immunotherapies. Unfortunately, both cGAMP and CpG have poor pharmacokinetics and limited clinical applicability due to various factors. For example, endogenous high concentrations of degradation enzymes quickly degrade these immunostimulatory molecules in vivo, limiting their usefulness.

Therefore, there is a significant need for the development of new materials that allow for the sustained exposure of vaccine and/or immunotherapy components, particularly of multiple compounds of various sizes, to the immune system. Described herein are methods, apparatuses and compositions that address these and other issues.

### BRIEF SUMMARY

Described herein are methods, apparatuses and compositions, e.g., kits, for introducing antigenic material, including antigens and adjuvants, into a subject. These methods, apparatuses and compositions can be particularly useful for creating and maintaining a high local concentration of adjuvants (and antigen) to establish an inflammatory niche, while also releasing the adjuvant (and antigen) cargo slowly over time to prolong their exposure to immune cells.

The development of an effective vaccine that can be rapidly manufactured and distributed worldwide would be beneficial to mitigate the devastating health and economic impacts of pandemics such as COVID-19, caused by SARS-COV-2. The receptor-binding domain (RBD) of the SARS-COV-2 spike protein, which mediates host cell entry of the virus, is an appealing antigen for subunit vaccines because it is easy to manufacture and highly stable. Moreover, RBD is a target for neutralizing antibodies and robust cytotoxic T lymphocyte responses. Unfortunately, RBD is poorly immunogenic compared to other antigens. Subunit vaccines are commonly formulated with adjuvants to enhance the immunogenicity, but most common adjuvant combinations have not been sufficient to improve RBD immunogenicity and none have afforded protection in a single-dose RBD vaccine. Here we show that delivering an RBD subunit vaccine in an injectable hydrogel increases total anti-RBD IgG titers compared to bolus administration of the vaccines. Notably, a SARS-COV-2 spike-pseudotyped lentivirus neutralization assay revealed neutralizing antibodies in all mice after a single hydrogel vaccine injection comprising clinically-approved adjuvants Aalum and CpG. Together, these results suggest that extending the exposure to RBD subunit vaccines significantly enhances the immunogenicity of RBD and induces neutralizing humoral immunity following a single immunization.

In one aspect, the disclosure provides a vaccine delivery system. The vaccine delivery system includes a hydrogel having a polymer non-covalently crossed-linked with a plurality of nanoparticles. The vaccine delivery system further includes a dinucleotide adjuvant encapsulated in the hydrogel. The vaccine delivery system further includes an antigen encapsulated in the hydrogel.

In another aspect, the disclosure provides a method for inducing an immune response against the antigen of any of the vaccine delivery systems disclosed herein in a subject. The method includes administering to the subject a therapeutically effective amount of the vaccine delivery system.

In another aspect, the disclosure provides a method of preventing or treating a disease in a subject. The method includes administering to the subject a therapeutically effective amount of any of the vaccine delivery systems disclosed herein.

In another aspect, the disclosure provides a method for delivering a vaccine to a subject. The method includes mixing a first solution having HPMC-C12 in a first receptacle with a second solution having PEG-PLA, an antigen, and a dinucleotide adjuvant in a second receptacle, to thereby form a homogenous solid-like hydrogel. The method further includes shearing the hydrogel through a syringe to form a shear-thinned gel. The method further includes delivering the hydrogel into an interior of the subject and forming a solid-like gel antigen and nucleotide adjuvant depot.

In another aspect, the disclosure provides a pharmaceutical agent kit including a first receptacle having a polymer. The pharmaceutical agent kit further includes a second receptacle having a nanoparticle, a dinucleotide adjuvant, and an antigen. The pharmaceutical reagent kit further includes a connector piece configured to fluidically connect the first receptacle with the second receptacle. The pharmaceutical reagent kit further includes an instructional material.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings.

FIG. 1 is a schematic illustration showing the entire SARS-COV-2 virus (˜40 nm), the spike trimer on its surface (˜7.5 nm), and its receptor-binding domain (˜5 nm).

FIG. 2 is a graph showing that RBD expression levels greatly exceed (˜100×) spike trimer expression levels in vaccine cargo. The bars of the graph represent the range of expression levels found in the literature.

FIG. 3 is a schematic illustration showing that larger 30-100 nm particles drain efficiently to lymph nodes and are retained there while smaller particles like RBD are not. Small, hydrophilic species like RBD suffer from poor pharmacokinetics.

FIG. 4 is a schematic illustration showing the combination of dodecyl-modified hydroxypropylmethylcellulose (HPMC-C12) with poly(ethylene glycol)-b-poly(lactic acid) (PEG-PLA) and vaccine cargo (RBD, CpG, and Alum) to form polymer-nanoparticle PNP hydrogels suitable for subcutaneous delivery of RBD and combinations of clinically de-risked adjuvants. Dynamic, multivalent noncovalent interactions between the polymer and nanoparticles lead to physical crosslinking within the hydrogel that behaves like a molecular Velcro.

FIG. 5 presents a series of photographs showing HPMC-C12 loaded into one syringe (left syringe in photographs (i)-(iii)) and the NP solution and vaccine components loaded into the other (right syringe in photographs (i)-(iii)). By connecting the syringes with an elbow (i) and rapidly mixing (ii), a homogenous, solid-like gel is formed (iii). The gel is then easily injected through a 21-gauge needle (iv) before self-healing and reforming a solid depot (v) in the subcutaneous space.

FIG. 6 is a graph plotting the frequency-dependent oscillatory shear rheology of a PNP hydrogel with or without Alum. The data show that rheological properties of PNP hydrogels allow for easy injection.

FIG. 7 is a graph plotting the shear-dependent viscosities of PNP hydrogels with or without Alum. The data show that rheological properties of PNP hydrogels allow for easy injection.

FIG. 8 is a graph plotting oscillatory amplitude sweeps of PNP hydrogels with or without Alum. The yield stresses were determined by the crossover points and are both around 1300 Pa. The data show that rheological properties of PNP hydrogels allow for easy injection.

FIG. 9 is a graph plotting step-shear measurements of hydrogels with or without Alum over three cycles of alternating high shear (gray; 10 s⁻¹) and low shear (white; 0.1 s⁻¹) rates. The data show that rheological properties of PNP hydrogels allow for easy injection.

FIG. 10 is a graph plotting the percent of CpG retained in a hydrogel in a glass capillary in vitro release study over time. Each point in the graph represents a separate hydrogel (n=3). The points were fit with a one phase-decay in GraphPad Prism and the half-life of release was determined. The data show that material properties of PNP hydrogels allow for slow release of vaccine cargo.

FIG. 11 is a graph plotting the percent of RBD retained in the same hydrogels as in the graph of FIG. 10. Each point in the graph represents a separate hydrogel (n=3). The points were fit with a linear fit in GraphPad Prism and the half-life of release was determined. The data show that material properties of PNP hydrogels allow for slow release of vaccine cargo.

FIG. 12 shows representative images demonstrating the different duration of release of Alexa-fluor 647-labeled RBD antigen given as a bolus or gel subcutaneous immunization over 18 days. The data show that material properties of PNP hydrogels allow for subcutaneous depot formation and slow release of vaccine cargo.

FIG. 13 is a graph plotting fluorescent signal from Alexa-fluor 647-labeled RBD (representative images shown in FIG. 12) for 3 weeks following immunization as determined by an In Vivo Imaging System (IVIS) (n=5). The points were fit with a one phase-decay in GraphPad Prism and the half-lives were determined. Data is shown as mean+/−SEM. The data show that material properties of PNP hydrogels allow for slow release of vaccine cargo.

FIG. 14 is a schematic illustration of a timeline of mouse immunizations and blood collection for different assays. Mice were immunized on day 0 and at week 8. Serum was collected weekly to determine IgG titers. IgM titers were assessed at week 1 (as shown in FIG. 40). IgG1, IgG2b, IgG2c titers were quantified and neutralization assays were conducted on week 4 and week 12 serum.

FIG. 15 is a graph plotting anti-RBD IgG ELISA titers before and after boosting (arrow) of several controls and the CpG+Alum+Gel group of interest. P values listed were determined using a 2-way ANOVA with Tukey's multiple comparisons test. P values for comparisons between the CpG+Alum+Gel group and all other groups for day 28 and day 84 are shown above the points. The data show that the hydrogel RBD vaccine increases antibody titers compared to bolus vaccine.

FIG. 16 is a graph plotting anti-RBD IgG1 titers from serum collected 4 weeks after mice were boosted. P values listed were determined using a one-way ANOVA with Tukey's multiple comparisons between the CpG+Alum+Gel group and each control group. The data show that the hydrogel RBD vaccine increases antibody titers compared to bolus vaccine.

FIG. 17 is a graph plotting anti-RBD IgG2b titers from serum collected 4 weeks after mice were boosted. P values listed were determined using a one-way ANOVA with Tukey's multiple comparisons between the CpG+Alum+Gel group and each control group. The data show that the hydrogel RBD vaccine increases antibody titers compared to bolus vaccine.

FIG. 18 is a graph plotting anti-RBD IgG2c titers from serum collected 4 weeks after mice were boosted. P values listed were determined using a one-way ANOVA with Tukey's multiple comparisons between the CpG+Alum+Gel group and each control group. The data show that the hydrogel RBD vaccine increases antibody titers compared to bolus vaccine.

FIG. 19 is a graph plotting the ratio of Anti-RBD IgG2c to IgG1 post-boost titers. Lower values (below 1) suggest a Th2 response or skewing towards a stronger humoral response.

FIG. 20 is a graph plotting anti-spike IgG titers determined 4 weeks after both the prime and boost immunizations using a spike-coated ELISA plate. P values for comparisons between the CpG+Alum+Gel group and all other groups for day 28 (top) and day 84 (bottom) are shown. All data are shown as individual mouse titer values (n=5) and the mean.

FIG. 21 is a schematic illustration of a timeline of mouse immunization and blood collection for different assays. A double-dose hydrogel (2× Gel) was administered a single time and no boost was given.

FIG. 22 is a graph plotting anti-RBD IgG ELISA titers over time. CpG+Alum and CpG+Alum+Gel groups were boosted at week 8 (arrows), but the 2× Gel group was not. Convalescent human serum collected from patients 9-10 weeks after the onset of symptoms is also shown for comparison. P values listed were determined using a 2way ANOVA with Tukey's multiple comparisons test on GraphPad Prism software. P values for comparisons between the 2× Gel group and all other groups for day 28 (top) and day 84 (bottom) are shown above the points. All data are shown as individual mouse or human titer values (n=5) and the mean. The data show that double-dose hydrogel induces a strong humoral response.

FIG. 23 is a graph plotting anti-spike IgG ELISA titers from serum collected at week 4 and week 12. Convalescent human serum titers are also shown. P values listed were determined using a 2way ANOVA with Tukey's multiple comparisons test on GraphPad Prism software. P values for comparisons between the 2× Gel group and all other groups for day 28 (top) and day 84 (bottom) are shown above the points. All data are shown as individual mouse or human titer values (n=5) and the mean. The data show that double-dose hydrogel induces a strong humoral response.

FIG. 24 is a graph plotting anti-RBD IgG1 titers from serum collected at week 4 week 12. P values listed were determined using a 2way ANOVA with Tukey's multiple comparisons test on GraphPad Prism software. P values for comparisons between the 2× Gel group and all other groups for day 28 (top) and day 84 (bottom) are shown above the points. All data are shown as individual mouse or human titer values (n=5) and the mean. The data show that double-dose hydrogel induces a strong humoral response.

FIG. 25 is a graph plotting anti-RBD IgG2c titers from serum collected at week 4 week 12. P values listed were determined using a 2way ANOVA with Tukey's multiple comparisons test on GraphPad Prism software. P values for comparisons between the 2× Gel group and all other groups for day 28 (top) and day 84 (bottom) are shown above the points. All data are shown as individual mouse or human titer values (n=5) and the mean. The data show that double-dose hydrogel induces a strong humoral response.

FIG. 26 is a graph plotting the ratio of IgG2c to IgG1 titers where lower values (below 1) suggest a Th2 response or skewing towards a stronger humoral response. Arrows separate pre- and post-boost data.

FIG. 27 is a graph plotting percent infectivity for Alum treatment at a range of serum dilutions as determined by a SARS-COV-2 spike-pseudotyped viral neutralization assay. Samples tested were from 4 weeks after the final immunization. Data are shown mean+/−SEM (n=5). Samples were run in technical duplicate on two separate occasions and values were averaged to determine the mean at each serum dilution.

FIG. 28 is a graph plotting percent infectivity for CpG+Alum treatment at a range of serum dilutions as determined by a SARS-COV-2 spike-pseudotyped viral neutralization assay. Samples tested were from 4 weeks after the final immunization. Data are shown mean+/−SEM (n=5). Samples were run in technical duplicate on two separate occasions and values were averaged to determine the mean at each serum dilution.

FIG. 29 is a graph plotting percent infectivity for CpG+Alum+Gel treatment at a range of serum dilutions as determined by a SARS-COV-2 spike-pseudotyped viral neutralization assay. Samples tested were from 4 weeks after the final immunization. Data are shown mean+/−SEM (n=5). Samples were run in technical duplicate on two separate occasions and values were averaged to determine the mean at each serum dilution.

FIG. 30 is a graph plotting percent infectivity for 2× Gel treatment at a range of serum dilutions as determined by a SARS-COV-2 spike-pseudotyped viral neutralization assay. Samples tested were from 4 weeks after the final immunization. Data are shown mean+/−SEM (n=5). Samples were run in technical duplicate on two separate occasions and values were averaged to determine the mean at each serum dilution.

FIG. 31 is a graph plotting percent infectivity for the same treatment groups as those of FIGS. 27-30 at a 1 in 50 serum dilution. Neutralizing titers of convalescent human serum collected 9-10 weeks after the onset of symptoms is also shown for comparison. Data are shown as individual mouse or human titer values (n=5) and the mean. P values listed were determined in GraphPad Prism software using a one-way ANOVA with Tukey's multiple comparison test. P values corresponding to comparisons between CpG+Alum+Gel or 2× Gel and other groups are shown. The data show that hydrogel RBD vaccines elicit neutralizing antibodies in mice.

FIG. 32 is a graph plotting IC50 values determined from the neutralization curves in FIGS. 27-30. Samples with neutralizing activity that was undetectable at a 1:50 dilution are excluded (all Alum and CpG+Alum samples). Data are shown as individual mouse or human titer values (n=5) and the mean. P values listed were determined in GraphPad Prism software using a one-way ANOVA with Tukey's multiple comparison test. P values corresponding to comparisons between CpG+Alum+Gel or 2× Gel and other groups are shown. The data show that hydrogel RBD vaccines elicit neutralizing antibodies in mice.

FIG. 33 is a graph plotting the relationship between IC50 values and Anti-RBD IgG titers for serum collected 4 weeks after the final immunization. Each point corresponds to a single mouse or human. The data shows that hydrogel RBD vaccines elicit neutralizing antibodies in mice.

FIG. 34 is a graph plotting anti-spike IgG ELISA titers from serum collected 4 weeks after the final immunization. Titers were determined for wildtype spike as well as UK and South African variants of spike. Data are shown as individual mouse or human titer values (n=5) and the mean. P values listed were determined in GraphPad Prism software using a one-way ANOVA with Tukey's multiple comparison test. P values from t tests comparing anti-spike titers with wildtype vs. South African variant of spike. The data show that hydrogel RBD vaccines elicit neutralizing antibodies in mice.

FIG. 35 is a schematic illustration of HPMC-C12 combined with PEG-PLA and vaccine cargo to form PNP hydrogels. Dynamic, multivalent noncovalent interactions between the polymer and NPs leads to physical crosslinking within the hydrogel that behaves like a molecular Velcro. For these studies, different combinations of class B CpG ODN1826 (CpG), Alhydrogel (Alum), Resiquimod (R848), Monophosphoryl lipid A (MPL), Quil-A (Sap), and the fatty-acid modified form of muramyl dipeptide (MDP) were used as adjuvants alongside the RBD antigen.

FIG. 36 is a graph plotting IFNα levels in serum collected 3 hours after the initial immunization as determined by ELISA (n=5). All treatments contain the adjuvants listed as well as the RBD antigen. The dotted lines show the detection limits of the assays. Individual values that each represent data from a single mouse are shown along with the mean for each group.

FIG. 37 is a graph plotting TNFα levels in serum collected 3 hours after the initial immunization as determined by ELISA (n=5). All treatments contain the adjuvants listed as well as the RBD antigen. The dotted lines show the detection limits of the assays. Individual values that each represent data from a single mouse are shown along with the mean for each group.

FIG. 38 is a graph plotting anti-RBD titers for all hydrogel vaccines tested. All vaccines include the adjuvants listed and the RBD antigen. The arrow shows the point at which mice were given a boost immunization. All data are shown as the mean+/−SEM (n=5).

FIG. 39 is a graph plotting anti-Spike IgG titers for all hydrogel vaccines tested. All vaccines include the adjuvants listed and the RBD antigen. The arrow shows the point at which mice were given a boost immunization. All data are shown as the mean+/−SEM (n=5).

FIG. 40 is a graph plotting Anti-RBD IgM titers of serum collected 7 days after immunization with each of the hydrogel vaccines tested. All vaccines include the adjuvants listed and the RBD antigen. All data are shown as the mean+/−SEM (n=5).

FIG. 41 is a graph plotting anti-RBD IgG1 titers 4 weeks after the prime (Day 28) and the boost (Day 84). All data are shown as the mean+/−SEM (n=5).

FIG. 42 is a graph plotting anti-RBD IgG2b titers 4 weeks after the prime (Day 28) and

the boost (Day 84). All data are shown as the mean+/−SEM (n=5).

FIG. 43 is a graph plotting anti-RBD IgG2c titers 4 weeks after the prime (Day 28) and the boost (Day 84). All data are shown as the mean+/−SEM (n=5).

FIG. 44 is a graph plotting the ratio of anti-RBD IgG2c to IgG1 post-boost (Day 84) titers. A value less than one indicates Th2 skewing and a stronger humoral response whereas a value over one indicates a stronger Th1 or cell-mediate response. All data are shown as the mean+/−SEM (n=5).

FIG. 45 is a graph plotting the percent infectivity as determined by a SARS-COV-2 spike-pseudotyped viral neutralization assay at a 1:250 serum dilution. Serum was collected 4 weeks after the boost immunization (Day 84). All points are the average of four normalized infectivity values obtained from two experimental replicates and data are shown as the cohort mean+/−SEM (n=5).

FIG. 46 is a graph plotting the ratio of anti-Spike IgG titers to anti-RBD IgG titers at day 28 and 84 for the primary groups of interest. Convalescent human serum is shown for comparison. All data are shown as mean+/−SEM (n=5).

FIG. 47 is a graph plotting the frequency of GC B cells from CD19+ cells collected from mice 2 weeks following hydrogel or bolus vaccination. All data are shown as mean+/−SEM (n=10) and P values from Mann-Whitney tests are shown.

FIG. 48 is a graph plotting the ratio of light zone (LZ) to dark zone (DZ) GC B cells collected from mice 2 weeks after immunization. All data are shown as mean+/−SEM (n=10) and P values from Mann-Whitney tests are shown.

FIG. 49 is a graph plotting the frequency of T follicular helper cells from CD4+ T cells collected from mice 2 weeks after immunization. All data are shown as mean+/−SEM (n=10) and P values from Mann-Whitney tests are shown.

FIG. 50 is a graph plotting CXCL13 concentration from serum collected 4, 6, and 8 weeks after immunization with CpG+Alum. Points were fit with a one-phase exponential decay on GraphPad Prism with a lower constraint set to 0. Each curve represents one mouse.

FIG. 51 is a graph plotting CXCL13 concentration from serum collected 4, 6, and 8 weeks after immunization with CpG+Alum+Gel. Points were fit with a one-phase exponential decay on GraphPad Prism with a lower constraint set to 0. Each curve represents one mouse.

FIG. 52 is a graph plotting the median half-life of decay from CXCL13 peak at week 4.

FIG. 53 is a photograph showing one step in a PNP hydrogel mixing process. The left syringe is loaded with HPMC-C12 and the right syringe is loaded with PEG-PLA NPs and vaccine cargo in PBS. After attaching the syringes with an elbow and being careful to exclude air, simple mixing yields a homogenous hydrogel.

FIG. 54 is a photograph showing a step in a PNP hydrogel injection process. The hydrogel (from FIG. 53) is readily injected through a 21-gauge needle. After injection, the hydrogel rapidly heals and forms a solid depot.

FIG. 55 is a graph plotting anti-RBD IgG ELISA endpoint titers before and after boosting (arrow) for alum bolus or gel immunizations. Immunizations were given at week 0 and week 8. SARS-COV-2 Spike RBD was the antigen (10 μg per dose) used for all groups and adjuvants are listed. Each point represents one mouse (n=5 per group) and bars show mean+/−SD.

FIG. 56 is a graph plotting anti-RBD IgG1 endpoint titers of post-boost, week 12 serum. Each point represents one mouse (n=5 per group) and bars show mean+/−SD.

FIG. 57 is a graph plotting anti-RBD IgG2c endpoint titers of post-boost, week 12 serum. Each point represents one mouse (n=5 per group) and bars show mean+/−SD.

FIG. 58 is a graph plotting the ratio of IgG2c to IgG1 titers where a higher ratio (greater than 1) suggests Th1 skewing and a lower ratio (less than 1) suggests Th2 skewing. Each point represents one mouse (n=5 per group) and bars show mean+/−SD.

FIG. 59 is a graph plotting relative infectivity using week 12 serum at a 1:250 dilution in a SARS-COV-2 spike-pseudotyped viral neutralization assay. Each point represents one mouse (n=5 per group) and error bars show mean+/−SD.

FIG. 60 is a graph plotting IC50 values derived from testing a wide range of serum dilutions in the same pseudotyped viral neutralization assay as that of FIG. 59. Only groups that showed a 50% reduction in infectivity in the initial screen of FIG. 59 were tested for FIG. 60. Each point represents one mouse (n=5 per group) and error bars show mean+/−SD.

FIG. 61 is a graph plotting anti-spike IgG endpoint titers of post-boost, week 12 serum. Titers against wildtype spike were tested as well as titers against two common variants, UK (B.1.1.7) and South Africa (B.1.351). Each point represents one mouse (n=5 per group) and error bars show mean+/−SD.

### DETAILED DESCRIPTION

Described herein are methods, apparatus, and compositions (e.g., kits) for introducing antigenic material, including antigens and adjuvants, into a subject. These methods, apparatuses and compositions can be particularly useful for creating and maintaining a high local concentration of adjuvants (and antigen) to establish an inflammatory niche, while also releasing the adjuvant (and antigen) cargo slowly over time to prolong their exposure to immune cells.

### Definitions

As used herein, the term “adjuvant” refers to a material for enhancing immunogenicity of an antigen. Immunostimulatory oligonucleotides (such as those including a CpG motif) can be used as adjuvants. Exemplary adjuvants suitable for use with the provided embodiments include 4-1BBL, aluminum including aluminum salts (e.g., amorphous aluminum hydroxyphosphate sulfate (AAHS), aluminum hydroxide, aluminum phosphate, potassium aluminum sulfate (Alum)), B7-1, B7-2, CD47, CD72, cyclic guanosine monophosphate adenosine monophosphate (2′3′-Cyclic GMP-AMP or cGAMP), cytosine phosphoguanine (CpG), dinucleotides, GM-CSF, IL-2, TNF-α, IFN-γ, G-CSF, LFA-3, OX-40L, Polyinosinic-polycytidylic acid (PIC), RANTES, and Toll-like receptor (TLR) agonists, such as TLR-7/8 agonists.

As used herein, the term “dinucleotide” refers to a compound composed of two nucleotides. A dinucleotide can be a cyclic dinucleotide (CDN). Examples of dinucleotides suitable for use with the provided embodiments include CpG and cGAMP.

As used herein, the term “nucleotide” refers to the basic building block of nucleic acid polymers. A nucleotide is an organic molecule made up of three subunits, a nucleobase, a five-carbon sugar (pentose), and a phosphate group.

As used herein, the term “pharmaceutical” refers to a substance used in the diagnosis, treatment, or prevention of disease and for restoring, correcting, modifying, or preventing organic functions.

### Systems and Devices

Described herein are immunomodulatory delivery systems, including a hydrogel having a polymer non-covalently crossed-linked with a plurality of nanoparticles (a PNP hydrogel); a first immunomodulatory cargo encapsulated in the hydrogel, wherein the first immunomodulatory cargo includes a nucleotide; and a second immunomodulatory cargo encapsulated in the hydrogel. Immunomodulatory delivery systems and methods contemplated for the systems and methods described herein include those described in WO 2020/072495, which is incorporated by reference in its entirety.

In some embodiments, the PNP hydrogels described herein can be made of one or more polymers, such as cellulose derivatives, such as hydroxypropylmethylcellulose (HPMC), hydroxyethylcellulose (HEC), methylcellulose (MC), carboxymethylcellulose (CMC), or hydroxypropylcellulose (HPC), or hyaluronic acid (HA) optionally modified with a hydrophobic moiety, such as hexyl (—C₆), octyl (—C₈), deceyl (—C₁₀), dodecyl (—C₁₂), phenyl (Ph), adamantyl, tetradecyl (—C₁₄), oleyl, or cholesterol (e.g., 5-30% modification, such as 5-25% modification, such as approximately 10-15% or 25%). In one specific embodiment, HPMC is 10-15% modified with dodecyl. In another specific embodiment, HEC is 25% modified with dodecyl. In another specific embodiment, HEC is 10% modified with cholesterol. Further, the polymer can be mixed with nanoparticles, such as nanoparticles having a diameter of less than 100 nm, e.g., less than 90 nm, less than 80 nm, less than 70 nm, less than 60 nm, less than 50 nm, less than 40 nm, less than 30 nm, less than 20 nm, or less than 10 nm. In some embodiments, the nanoparticles have an average diameter between 10 nm and 100 nm, e.g., between 20 nm and 40 nm, between 25 nm and 45 nm, between 30 nm and 50 nm, between 35 nm and 55 nm, or between 40 nm and 60 nm. In some embodiments, the nanoparticles have a diameter that is approximately 40 nm. The nanoparticles can be core-shell nanoparticles with hydrophobic cores, such as poly(ethylene glycol)-block-poly(lactic acid) (PEG-PLA) or poly(ethyleneglycol)-block-poly(caprolactone) (PEG-PCL) nanoparticles.

The first or second immunomodulatory cargo can include an adjuvant, such as a nucleotide adjuvant.

The first or second immunomodulatory cargo (or a separate compound) can include another immune therapy, such as anti-PD1 antibodies, anti-PDL1 antibodies, anti-CD47 antibodies, anti-CD40 antibodies, anti-CD28 antibodies, toll-like receptor agonists, IL2 cytokines, IL12 cytokines, IL15 cytokines, GMCSF cytokines, chemokines, bispecific antibodies, bispecific T-cell engagers, or a combination thereof. Delivering the immunomodulatory delivery system can include injecting the immunomodulatory delivery system into the patient.

The immunomodulatory delivery system can further include one or more excipients, e.g., substances that aid the administration of the active agent to a cell, an organism, or a subject. A carrier or excipient can be included in the provided pharmaceutical compositions of the invention if causing no significant adverse toxicological effect on the patient. Non-limiting examples of pharmaceutically acceptable carriers include water, sodium chloride (NaCl), normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The pharmaceutically acceptable carrier can comprise or consist of one or more substances for providing the formulation with stability, sterility and isotonicity, e.g., antimicrobial preservatives, antioxidants, chelating agents and buffers. The pharmaceutically acceptable carrier can comprise or consist of one or more substances for preventing the growth or action of microorganisms, e.g., antimicrobial and antifungal agents, such as parabens, chlorobutanol, phenol, sorbic acid and the like. The pharmaceutically acceptable carrier can comprise or consist of one or more substances for providing the formulation with a more palatable or edible flavor . . . .

### Methods

In another aspect, a method for inducing an immune response against an antigen in a subject is disclosed. The method includes administering to the subject a therapeutically effective amount of any of the pharmaceutical compositions disclosed herein and described in further detail above, e.g., by using any of the disclosed immunomodulatory delivery systems.

As used herein, the term “subject” refers to a vertebrate, preferably a mammal, more preferably a human. Mammals include, but are not limited to, murines, mice, rats, simians, humans, farm animals, sport animals, and pets. Tissues, cells and their progeny of a biological entity obtained in vivo or cultured in vitro are also encompassed. In some embodiments, the subject is human. In some embodiments, the subject is male. In some embodiments, the subject is female. In some embodiments, the subject is an adult. In some embodiments, the subject is an adolescent. In some embodiments, the subject is a child. In some embodiments, the subject is above 60, 70, or 80 years of age.

As used herein, the term “therapeutically effective amount” refers to the amount of an immunomodulatory delivery system described herein that is sufficient to effect beneficial or desired results. The therapeutically effective amount can vary depending upon one or more of the subject and disease condition being treated, the weight and age of the subject, the severity of the disease condition, the immune status of the subject, the manner of administration and the like, which can readily be determined by one of ordinary skill in the art. The specific amount can further vary depending on one or more of the particular agent chosen, the target cell type, the location of the target cell in the subject, the dosing regimen to be followed, whether the provided delivery system or composition is administered in combination with other compounds, and the timing of administration.

For the purposes herein an effective amount is determined by such considerations as may be known in the art. The amount must be effective to achieve the desired therapeutic effect in a subject suffering from a disease such as an infectious disease or cancer. The desired therapeutic effect can include, for example, amelioration of undesired symptoms associated with the disease, prevention of the manifestation of such symptoms before they occur, slowing down the progression of symptoms associated with the disease, slowing down or limiting any irreversible damage caused by the disease, lessening the severity of or curing the disease, or improving the survival rate or providing more rapid recovery from the disease. Further, in the context of prophylactic treatment the amount can also be effective to prevent the development of the disease.

In some embodiments, the provided method further includes the step of providing to the subject a diagnosis and/or the results of treatment.

In another aspect, a method for preventing or treating a disease in a subject is disclosed. The method includes administering to the subject a therapeutically effective amount of any of the pharmaceutical compositions or immunomodulatory delivery systems disclosed herein and described in further detail above. As used herein, the term “treating” refers to an approach for obtaining beneficial or desired results including, but not limited to, a therapeutic benefit and/or a prophylactic benefit. “Therapeutic benefit” means any therapeutically relevant improvement in or effect on one or more diseases, conditions, or symptoms under treatment. Therapeutic benefit can also mean to effect a cure of one or more diseases, conditions, or symptoms under treatment. Furthermore, therapeutic benefit can also refer to an increase in survival. For prophylactic benefit, the compositions can be administered to a subject at risk of developing a particular disease, condition, or symptom, or to a subject reporting one or more of the physiological symptoms of a disease, even though the disease, condition, or symptom may not yet be present.

In some embodiments, the disease prevented or treated with the provided method can be an infectious disease or cancer. The infectious disease can be any of those described herein. The infectious disease can be caused by, for example, a bacterial infection, a viral infection, a fungal infection, a protozoal infection, a helminthic infection, or a combination thereof. In some embodiments, the treating of the disease in the subject includes decreasing or eliminating one or more signs or symptoms of the disease. In some embodiments, the infection is a SARS-COV-2 infection.

As shown herein, cGAMP or CpG with Alhydrogel in PNP hydrogels with a specific antigen elicits a very robust humoral immune response. Other combinations of different adjuvants (either multiple dinucleotide-based adjuvants or a combination of adjuvant types) with an antigen can be loaded to induce an even stronger response. Furthermore, the immunomodulatory delivery system, adjuvants, and hydrogel can be combined with other protein antigen(s) or larger antigen scaffolds to tailor the response.

The materials described herein have been optimized for delivery of adjuvants, such as cGAMP, to supplement cancer immunotherapy. Although published reports indicate that endosomolytic polymerosomes delivering cGAMP improved results of immune checkpoint blockade, the treatment required intratumoral administration. In order for cGAMP to be an effective adjuvant for subunit vaccines and for cancers that are hard to access or metastatic, prolonging cGAMP stability and improving the pharmacokinetics is helpful, because the cGAMP cannot always simply be administered at the site that it needs to act. It may need to be administered remote from the site. CpG has similarly been pursued as an adjuvant for subunit vaccines and cancer immunotherapies. Nanodiscs, DNA hydrogels, and microneedle patches are materials that have been used to enhance stability and delivery of CpG. The PNP hydrogels described herein are advantageous for delivery of cGAMP, CpG, and other adjuvants because they are injectable, they can be loaded with a wide array of different classes of molecules, and they require mild and simple synthesis methods.

Some embodiments provide include a method for delivering a pharmaceutical to a subject. The pharmaceutical can be a vaccine or an immunotherapy. The methods can include combining a first solution from a first receptacle with a second solution from a second receptable through a connector. The methods may include mixing a first solution comprising a polymer in a first receptacle with a second solution comprising nanoparticles, an antigen, and a nucleotide adjuvant in a second receptacle, to thereby form a homogenous solid-like hydrogel. Steps in the method may include shearing the hydrogel through a syringe to form a shear-thinned gel; and delivering the hydrogel into an interior of a patient and forming a solid-like gel antigen and nucleotide adjuvant depot. The first solution may be any of the polymers described herein. In some embodiments, the polymer is a cellulose derivative, such as hydroxypropylmethylcellulose (HPMC). The second solution may include nanoparticles, such as poly(ethylene glycol)-bpoly(lactic acid) (PEG-PLA).

### Kits

In another aspect, a kit is provided. The kit includes any of the pharmaceutical compositions or immunomodulatory delivery systems disclosed herein and described in further detail above. In some embodiments, the kit is useful for inducing an immune response against a targeted antigen.

The provided kit can be packaged in a way that allows for safe or convenient storage or use. The kit can be packaged, for example, in a box or other container having a lid. Typically, the provided kit includes one or more containers, e.g., a first receptacle and/or a second receptacle. The first and/or second receptacles can include syringes (e.g., standard or non-standard syringes, such as 1-mL syringes, 2-mL syringes, etc. that can have a luer ending). The connector can be an elbow-shaped tube to fluidically connect the first and/or second receptacles, such as with one or more mating luer connectors. Other connections are also contemplated. The connector can include a valve or other divider configured to maintain the contents of the first solution separate from the contents of the second solution until it is time to mix the contents. After mixing, the mixed contents can be collected into a first of the receptacles and the connector removed, such as by unscrewing the connector from the first receptacle. An injection needle or catheter connection can be placed on an end of the receptacle for use in delivering the mixed pharmaceutical to an interior of a patient, such as to a muscle or in or adjacent a tumor site. The final pharmaceutical (hydrogel) encapsulates vaccine components (including the cGAMP, CpG, or other adjuvants) efficiently, is injectable, and can co-deliver diverse cargo over prolonged timeframes.

In some embodiments, the kit further includes instructions for use, e.g., containing directions for the practice of a provided method. While the instructional materials typically include written or printed materials they are not limited to such. Any medium capable of storing such instructions and communicating them to an end user is contemplated by this invention. Such media include, but are not limited to electronic storage media, e.g., magnetic discs, tapes, cartridges, chips; optical media, e.g., CD-ROM; and the like. Such media can include addresses to internet sites that provide such instructional materials.

### Embodiments

The following embodiments are contemplated. All combinations of features and embodiment are contemplated.

Embodiment 1: A vaccine delivery system, comprising: a hydrogel comprising a polymer non-covalently crossed-linked with a plurality of nanoparticles; a dinucleotide adjuvant encapsulated in the hydrogel; and an antigen encapsulated in the hydrogel.

Embodiment 2: An embodiment of embodiment 1, wherein the dinucleotide adjuvant comprises CpG.

Embodiment 3: An embodiment of embodiment 1, wherein the dinucleotide adjuvant comprises a cyclic dinucleotide.

Embodiment 4: An embodiment of embodiment 3, wherein the cyclic dinucleotide comprises cGAMP.

Embodiment 5: An embodiment of any of the embodiments of embodiment 1-4, wherein the antigen comprises a receptor binding domain (RBD) of a virus.

Embodiment 6: An embodiment of embodiment 5, wherein the virus is a SARS-COV virus, a SARS-COV-2 virus, or a MERS-COV virus.

Embodiment 7: An embodiment of any of the embodiments of embodiment 1-6, wherein the polymer comprises hydroxypropylmethylcellulose (HPMC), or a derivative thereof.

Embodiment 8: An embodiment of any of the embodiments of embodiment 1-7, wherein the nanoparticles are polymeric nanoparticles.

Embodiment 9: An embodiment of embodiment 8, wherein the polymeric nanoparticles comprise poly(ethylene glycol)-bpoly(lactic acid) (PEG-PLA).

Embodiment 10: An embodiment of any of the embodiments of embodiment 1-9, further comprising: an aluminum or aluminum salt adjuvant encapsulated in the hydrogel.

Embodiment 11: An embodiment of embodiment 10, wherein the aluminum or aluminum salt adjuvant comprises aluminum hydroxide.

Embodiment 12: An embodiment of any of the embodiments of embodiment 1-11, further comprising: one or more additional adjuvants selected from the list consisting of Resiquimod (R848), Monophosphoryl lipid A (MPL), Quil-A (Sap), and the fatty-acid modified form of muramyl dipeptide (MDP).

Embodiment 13: A method for inducing an immune response against the antigen of the vaccine delivery system of any of the embodiments of embodiment 1-12 in a subject, the method comprising: administering to the subject a therapeutically effective amount of the vaccine delivery system.

Embodiment 14: An embodiment of embodiment 13, wherein the immune response comprises increased production of IgG antibodies.

Embodiment 15: An embodiment of embodiment 14, wherein the immune response comprises increased production of IgG1 antibodies.

Embodiment 16: An embodiment of embodiment 14 or 15, wherein the immune response comprises increased production of IgG2b antibodies.

Embodiment 17: An embodiment of any of the embodiments of embodiment 14-16, wherein the immune response comprises increased production of IgG2c antibodies.

Embodiment 18: An embodiment of any of the embodiments of embodiment 14-17, wherein the ratio of the concentration of IgG2c to the concentration of IgG1 in a serum sample from the subject taken after the administering is less than 0.3:1.

Embodiment 19: A method of preventing or treating a disease in a subject, the method comprising: administering to the subject a therapeutically effective amount of the vaccine delivery system of any of the embodiments of embodiment 1-12.

Embodiment 20: An embodiment of embodiment 19, wherein, subsequent to the administering, the dinucleotide adjuvant and the antigen release from the hydrogel into the subject at substantially the same rate.

Embodiment 21: An embodiment of embodiment 19 or 20, wherein the disease is COVID-19.

Embodiment 22: An embodiment of any of the embodiments of embodiment 19-21, wherein administering the vaccine delivery system comprises injecting the vaccine delivery system into the subject.

Embodiment 23: A method for delivering a vaccine to a subject, the method comprising: mixing a first solution comprising HPMC-C12 in a first receptacle with a second solution comprising PEG-PLA, an antigen, and a dinucleotide adjuvant in a second receptacle, to thereby form a homogenous solid-like hydrogel; shearing the hydrogel through a syringe to form a shear-thinned gel; and delivering the hydrogel into an interior of the subject and forming a solid-like gel antigen and nucleotide adjuvant depot.

Embodiment 24: An embodiment of embodiment 23, wherein at least the first receptacle or the second receptacle comprises the syringe.

Embodiment 25: An embodiment of embodiment 23 or 24, wherein the solid-like gel antigen and dinucleotide adjuvant depot is configured to release antigen and dinucleotide in the subject for at least two weeks.

Embodiment 26: An embodiment of any of the embodiments of embodiment 23-25, wherein the second solution further comprises one or more additional adjuvants selected from the list consisting of an aluminum or aluminum salt, Resiquimod (R848), Monophosphoryl lipid A (MPL), Quil-A (Sap), and the fatty-acid modified form of muramyl dipeptide (MDP).

Embodiment 27: A pharmaceutical agent kit comprising: a first receptacle comprising a polymer; a second receptacle comprising a nanoparticle, a dinucleotide adjuvant, and an antigen; a connector piece configured to fluidically connect the first receptacle with the second receptacle; and an instructional material.

Embodiment 28: An embodiment of embodiment 27, wherein the polymer comprises dodecyl-modified hydroxypropylmethylcellulose (HPMC-C12).

Embodiment 29: An embodiment of embodiment 27 or 28, wherein the nanoparticle comprises poly(ethylene glycol)-bpoly(lactic acid) (PEG-PLA).

Embodiment 30: An embodiment of any of the embodiments of embodiment 27-29, wherein the first receptacle and the second receptacle comprise syringes.

Embodiment 31: An embodiment of any of the embodiments of embodiment 27-30, wherein the second receptacle comprises a receptor binding domain (RBD) of a virus.

### Examples

The present disclosure will be better understood in view of the following non-limiting examples. The following examples are intended for illustrative purposes only and do not limit in any way the scope of the present invention. The examples demonstrate that, for example, hydrogel-based slow release of receptor-binding domain subunit vaccine elicits neutralizing antibody responses against SARS-COV-2.

The COVID-19 pandemic has had devastating health and economic impacts globally since SARS-COV-2 first infected humans in 2019. In less than two years, COVID-19 has caused over 2.4 million deaths globally, including over 500,000 deaths in the United States alone. Although behavioral and contact tracing interventions have slowed the spread and vaccines are becoming available in some regions, case numbers remain high in many parts of the world. Continued spread of SARS-COV-2 are expected to be particularly harmful in regions that have limited resources and access to healthcare. High rates of asymptomatic transmission and the lack of effective treatments has made the virus difficult to contain. Deployment of effective vaccines is therefore a critical global health priority toward managing or ending the COVID-19 pandemic. Additionally, COVID-19 has reinforced the importance of developing vaccine platforms that can be rapidly adapted to respond to new pathogens and future pandemics.

There are different SARS-COV-2 vaccine candidates at various stages of development and clinical testing including novel platforms based on DNA or mRNA. Notably, the COVID-19 mRNA vaccines made by Pfizer/BioNTech and Moderna, have been approved by the FDA. While mRNA vaccines are expected to play a significant role in mitigating effects of the pandemic in areas such as the United States and much of Europe, they face manufacturing and distribution limitations that constrain their impact in low-resource settings. Subunit vaccines (e.g., those containing a fragment of a pathogen rather than a whole pathogen) based on recombinant proteins may be more stable and less reliant on the cold chain, making them cheaper and easier to produce and distribute. Importantly, large-scale production capacity for subunit vaccines already exists in many regions of the world. Similar to mRNA vaccines, subunit vaccines may be safer than live attenuated vaccines and therefore may be recommended for older and immunocompromised populations. COVID-19 and future pandemics might not be contained until people all around the globe are protected from the virus and thus cost, stability, and ease of manufacturing and distribution are qualities to consider in vaccine development.

The receptor-binding domain (RBD) of the spike protein that coats the surface of SARS-CoV-2 is an appealing target antigen for COVID-19 subunit vaccines. RBD is the portion of the spike protein that binds to the human angiotensin converting enzyme 2 (ACE2) receptor to mediate viral infection. RBD is more stable than the spike trimer and is manufactured using low-cost, scalable expression platforms. Remarkably, literature reports show that expression levels of RBD can be 100-times greater than expression levels of spike trimer as measured by mass of protein recovered. Recent considerations to halve COVID-19 vaccine doses, only provide one injection when two are recommended, or increase the time between doses highlight the need for greater vaccine scalability. Notably, RBD is the target for many neutralizing antibodies that have been identified and is a sufficient source of T cell epitopes for a potent cytotoxic T lymphocyte response. An analysis of antibodies produced by survivors of COVID-19 showed that a larger proportion of RBD-binding antibodies were neutralizing compared to those that bound spike outside of the RBD domain. Lastly, regions of spike outside of the RBD domain have been shown to induce antibody-dependent immune enhancement in non-human primates further supporting the use of RBD. Unfortunately, RBD is not highly immunogenic on its own. In this disclosure we demonstrate the rescue of the immunogenicity of RBD by slowly delivering the antigen together with potent clinically de-risked adjuvants from an injectable hydrogel.

Slow delivery of antigen(s) can result in a more potent humoral immune response. Previous work delivering an HIV antigen from an implantable osmotic pump led to 20-30 times higher titers compared to conventional bolus administration in non-human primates. Microneedle patches are a less-invasive alternative to osmotic pumps, but unfortunately, they often require harsh synthesis and loading processes that damage antigens. For this reason, the hydrogel platforms for vaccine delivery disclosed herein can be significantly advantageous. Although some hydrogels may be easy to make, provide improved vaccine cargo stability, and mimic human tissues, unfortunately, many hydrogels are covalently cross-linked, which may limit their injectability and loading of diverse cargo. To address these limitations, the provided injectable polymer nanoparticle (PNP) hydrogel can be loaded with a diverse range of vaccine cargo. These PNP hydrogel vaccines promote greater affinity maturation and generate a durable, robust humoral response.

Supplementing subunit vaccine antigens with one or more potent adjuvants can further enhance the immune response. As shown below, sustained exposure of RBD subunit vaccines comprising various clinically de-risked adjuvants within an injectable hydrogel depot increases total anti-RBD IgG titers when compared to the same vaccines administered as a bolus injection. Notably, a lentiviral SARS-COV-2 pseudovirus assay revealed neutralization after a single injection of the hydrogel-based vaccine comprising CpG and Alum described herein. The slow release of complete RBD subunit vaccines described herein significantly enhances the immunogenicity of RBD and induces neutralizing humoral immunity following a single immunization.

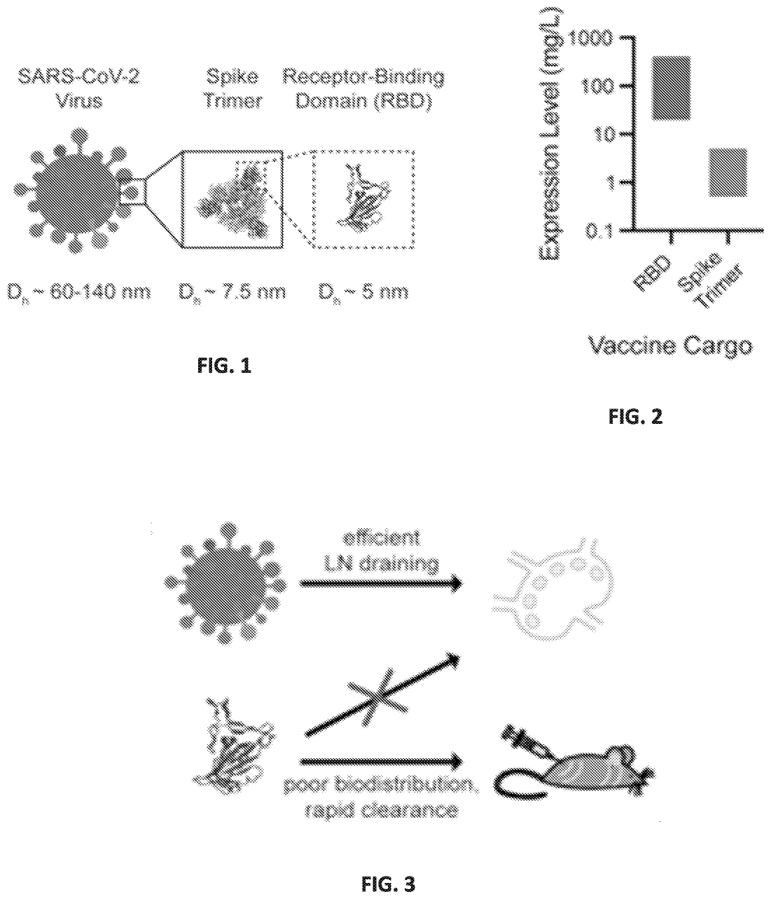

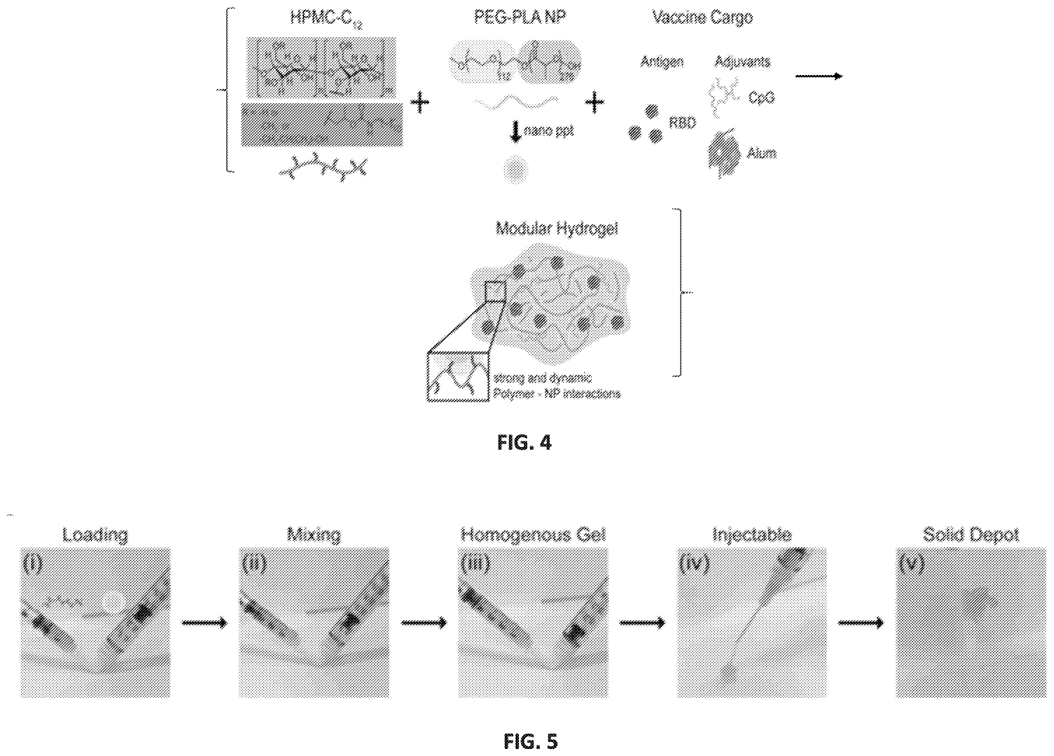

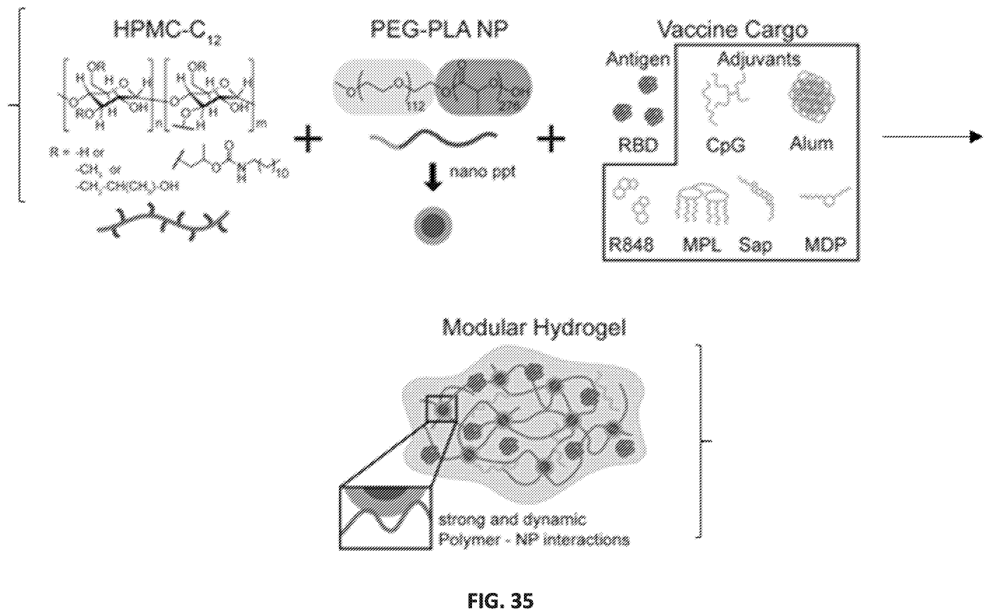

RBD was used as the antigen for all vaccine formulations described herein because of its high expression levels, ease of manufacturing, and stability (FIGS. 1 and 2). Due to RBD's small size, it does not drain to, or remain in, lymph nodes nearly as efficiently as does the SARS-COV-2 virus itself, limiting RBD's interaction with critical immune cells (FIG. 3). Small antigens like RBD often have poor pharmacokinetics, are quickly dispersed throughout the body (after delivery) and are cleared rapidly. In order to prolong RBD availability and interaction with immune cells, we generated a polymer-nanoparticle (PNP) hydrogel with RBD. Formulation of the polymer-nanoparticle (PNP) hydrogel is described in WO 2020/072495. PNP hydrogels form rapidly upon mixing of hydroxypropylmethylcellulose derivates (HPMC-C12) and biodegradable polymeric NPs made of poly(ethylene glycol)-b-poly(lactic acid) (PEG-PLA) (FIG. 4). By adding antigen and adjuvant(s) to the NP solution, vaccine components are readily incorporated into the aqueous phase of the hydrogel (FIG. 4). To boost the immunogenicity of our RBD vaccines, combinations of clinically de-risked adjuvants were incorporated. Described herein are results from a hydrogel vaccine comprising RBD, class B CpG ODN1826 (CpG), and Alhydrogel (Alum, aluminum hydroxide) (FIG. 4). Studies were also conducted with different combinations of toll-like receptor (TLR) and NOD-like receptor (NLR) agonists Resiquimod (R848), Monophosphoryl lipid A (MPL), Quil-A (Sap), and the fatty-acid modified form of muramyl dipeptide (MDP) (FIG. 35).

The PNP hydrogel encapsulates vaccine components efficiently, is injectable, and can co-deliver diverse cargo over prolonged timeframes. HPMC-C12 is loaded into one syringe and the NP solution and vaccine components are loaded into the other (FIG. 5). By connecting the syringes with an ‘elbow’ and mixing, a homogenous gel is formed (FIG. 5). The gel is then easily injected through a needle before self-healing and re-forming a solid depot under the skin (FIG. 5).

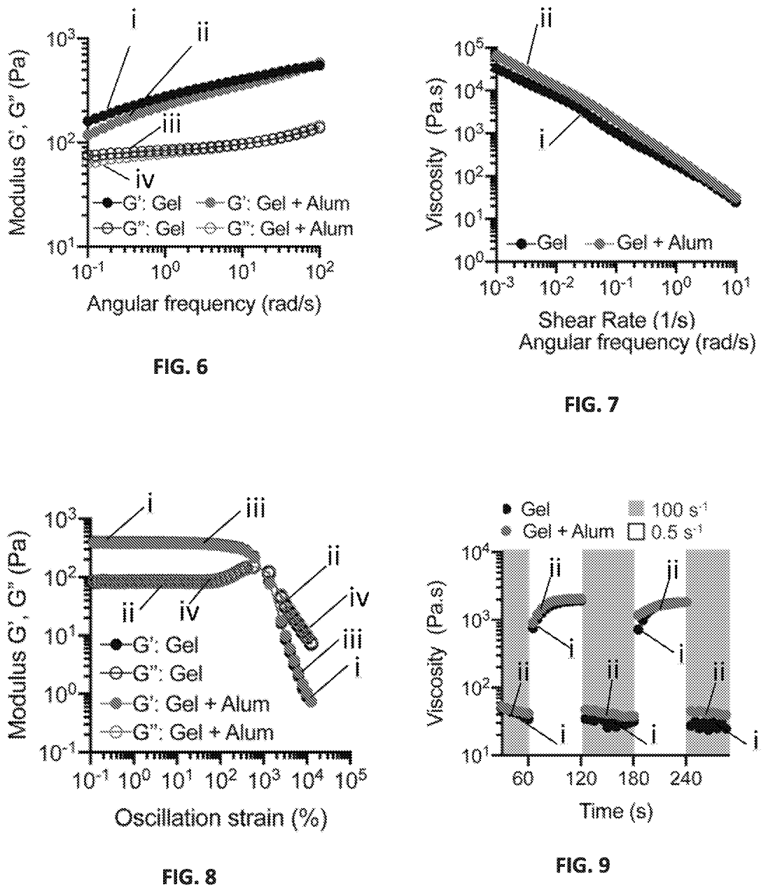

Rheological properties of the hydrogel that are indicative of injectability and depot formation were measured. We compared a PNP hydrogel without Alum and one with Alum determine if Alum did not interfere with the rheological properties previously observed. Frequency-dependent oscillatory shear experiments performed in the linear viscoelastic regime showed that the PNP hydrogels with and without Alum had nearly identical frequency responses (FIG. 6). For both formulations, the storage modulus (G′) remained above the loss modulus (G″) across a wide range of frequencies, meaning gels remained solid-like across this frequency range (FIG. 6).

Injectability depends on shearing properties of the hydrogel. A shear rate sweep showed that the viscosity of the hydrogels (with or without Alum) decreased several orders of magnitude as the shear rate increased, demonstrating the ability to shear-thin (FIG. 7). To assess yielding behavior of the hydrogels, a dynamic amplitude sweep was performed at a frequency of 10 rad/s. For both hydrogels, a yield stress of about 1300 Pa was measured at the crossover point of G′ and G″ (FIG. 8). Injectability was then tested by measuring the change in viscosity when alternating between a high shear rate (10 s⁻¹) and a low shear rate (0.1 s⁻¹) (FIG. 9). The viscosity of the hydrogels with and without Alum decreased by about two orders of magnitude under high shear (FIG. 9). This test of shear thinning followed by self-healing of the hydrogels mimics an injection through a needle (high shear rate) and the subsequent subcutaneous (SC) depot formation (low shear rate). These data demonstrate that a solid hydrogel depot will form and remain in the SC space after injection which allows for slow release of cargo over time.

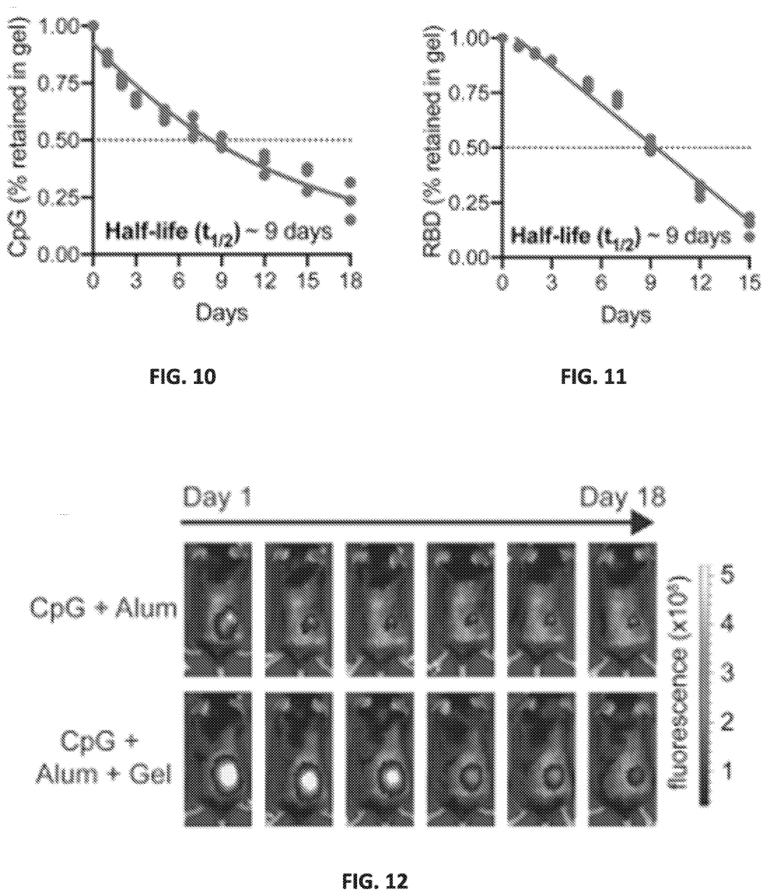

Previous research has shown that proteins and negatively charged molecules can adsorb to Alum. To test if the addition of Alum would therefore further slow the release of RBD and negatively charged CpG from the hydrogel, an in vitro capillary setup was used to quantify the release of CpG and RBD from the Alum-containing hydrogel over time. The hydrogel was injected into the bottom of a capillary tube and buffer was added above to provide a large sink for release. Tubes were incubated at 37° C. to mimic physiological conditions and the entire buffer sink was removed and replaced at each timepoint shown (FIGS. 10 and 11). CpG and RBD were released slowly from the gel with retention half-lives of about 9 days (FIGS. 10 and 11).

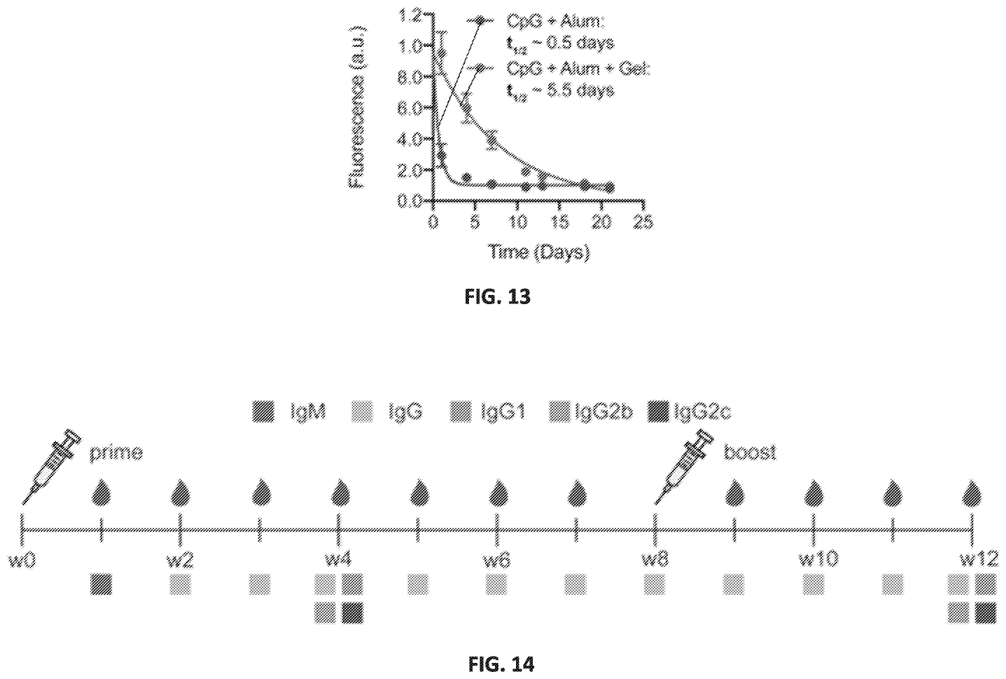

Since in vitro release studies are different from release within a living organism, we next quantified retention of RBD in mice following SC injection. RBD was conjugated to an Alexa Fluor 647 dye and CpG+Alum+Gel and CpG+Alum bolus treatments were prepared using the methods described for the following vaccine studies. The amount of RBD retained in the hydrogel over 18 days was monitored by fluorescence IVIS imaging. Labeled RBD from the bolus treatment was almost undetectable within about a week while RBD from the hydrogel treatment persisted for the duration of the study (FIG. 12). We fit fluorescence values over time with a one-phase exponential decay and determined that the half-life of RBD release was extended from about 0.5 days to 5.5 days when delivered in the hydrogel (FIG. 13). The half-life measured in vivo is within half an order of magnitude of the half-life measured in vitro. Without being bound to a particular theory, it is hypothesized that the decrease in half-life in vivo may be due to the fact that cells can actively transport RBD out of the hydrogel and in vivo proteolytic degradation of RBD can occur.

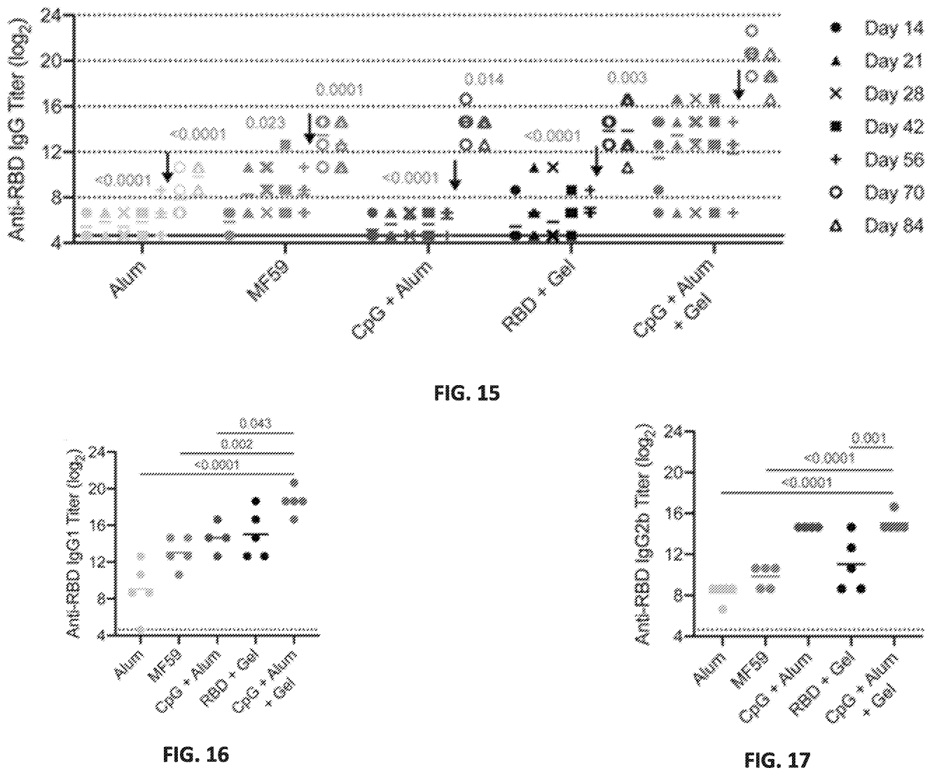

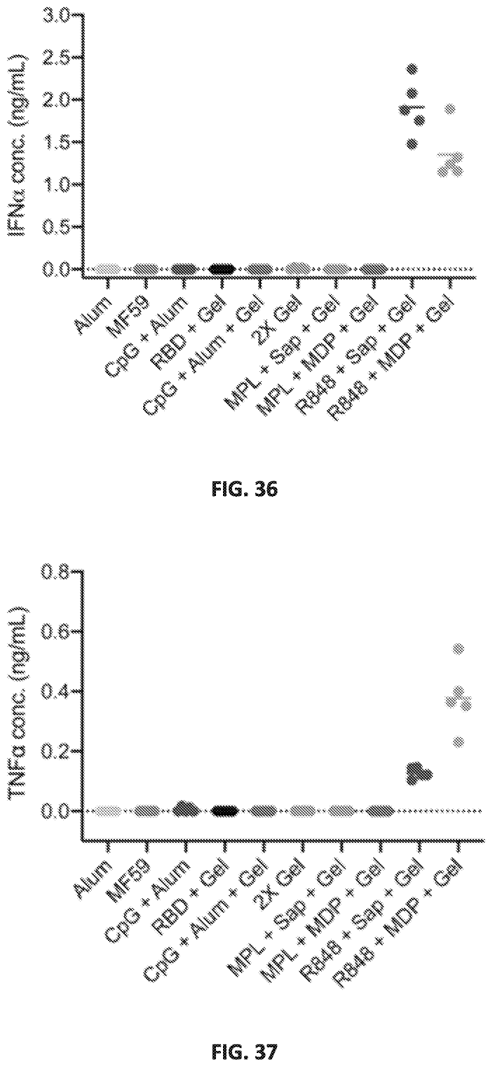

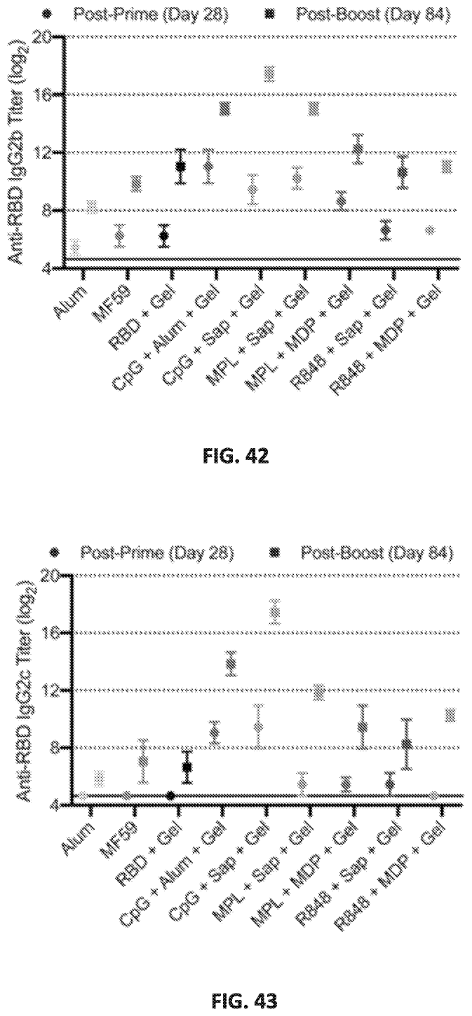

In order to evaluate whether RBD delivery in an adjuvanted hydrogel enhanced the humoral immune response, we quantified antigen-specific antibody titers over time in C57BL/6 mice (n=5 each). The hydrogel and bolus control were loaded with 10 μg of RBD. In addition to IgG antibody titers, we quantified different antibody classes and subclasses (IgM, IgG1, IgG2b, IgG2c) to assess the quality of the response as well as the anti-spike IgG antibody response and the acute cytokine response shortly after administration (FIGS. 14 and 36-43). Vaccines were administered on day 0 and mice were boosted with the original treatment on week 8. Serum was collected weekly and assays were run at the timepoints shown in FIG. 14. High systemic levels of certain cytokines is correlated with toxicity in mice and humans. We measured IFNα and TNFα concentrations at 3- and 24-hours post-immunization as a measure of toxicity for each formulation. The only treatments that led to detectable cytokine levels at 3 hours were R848+Sap+Gel and R848+MDP+Gel (FIGS. 14 and 15). The IFNα serum concentrations for these treatments were 1-2 ng/ml and the TNFα concentrations were below 0.5 ng/ml. These data suggest that the CpG+Alum+Gel and other treatments that did not include R848 were well-tolerated by this measure.

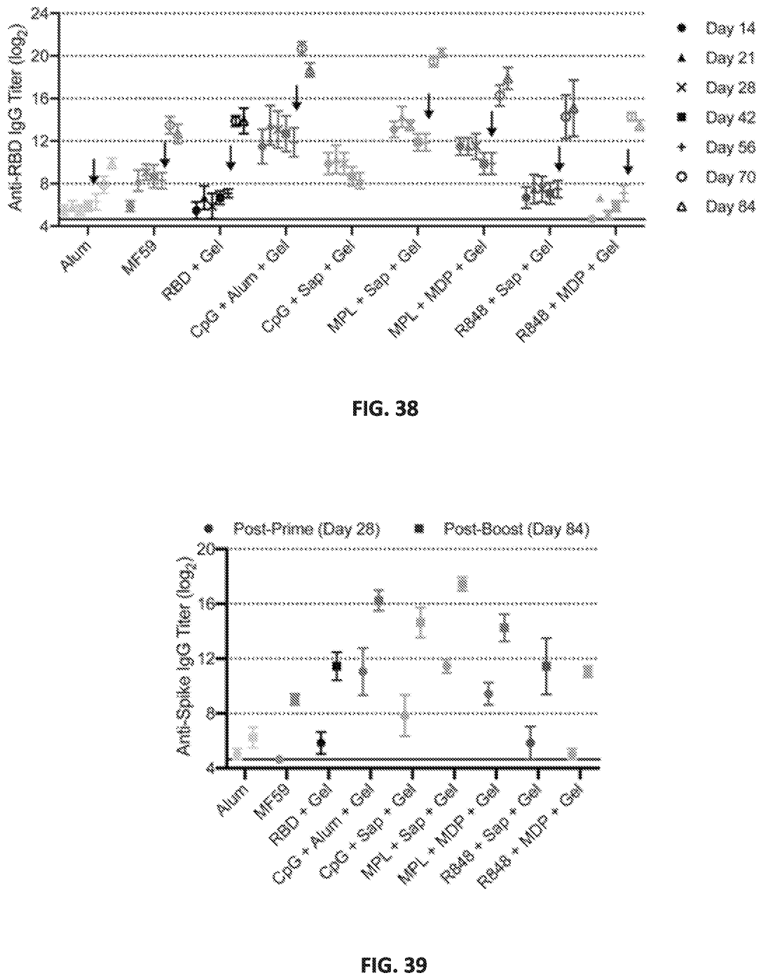

Both before and after boosting, mice treated with CpG and Alum in the PNP hydrogel (CpG+Alum+Gel) had higher total antigen-specific IgG antibody titers than Alum, MF59, CpG+Alum bolus control, and hydrogel with the RBD antigen only (RBD+Gel). After boosting, the CpG+Alum+Gel treatment led to titers that were ˜60 times greater than all controls including the bolus treatment that contained identical antigen and adjuvants (FIG. 15). As expected, there was a notable increase in titer across all groups following the boost. Additional gels containing RBD and additional adjuvant combinations were also tested. A hydrogel loaded with MPL, QuilA, and RBD (MPL+Sap+Gel) resulted in similar titers to the CpG+Alum+Gel treatment. Surprisingly, although R848-containing gels led to an increase in serum cytokines at early timepoints, these treatments were not as effective at inducing high titers (FIGS. 38 and 39).

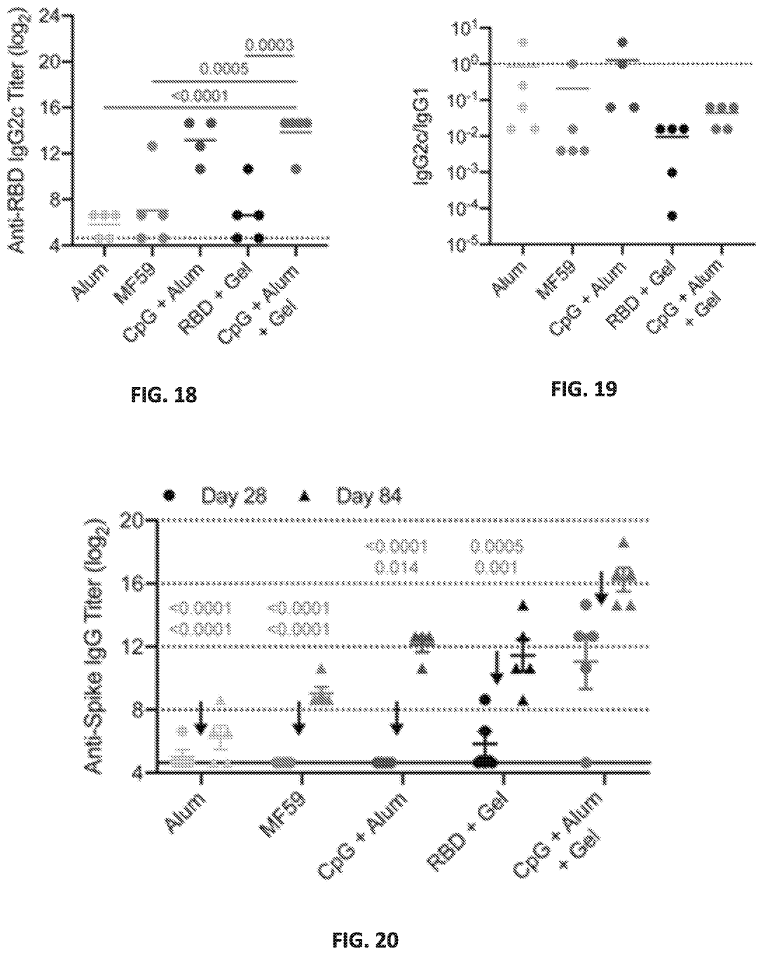

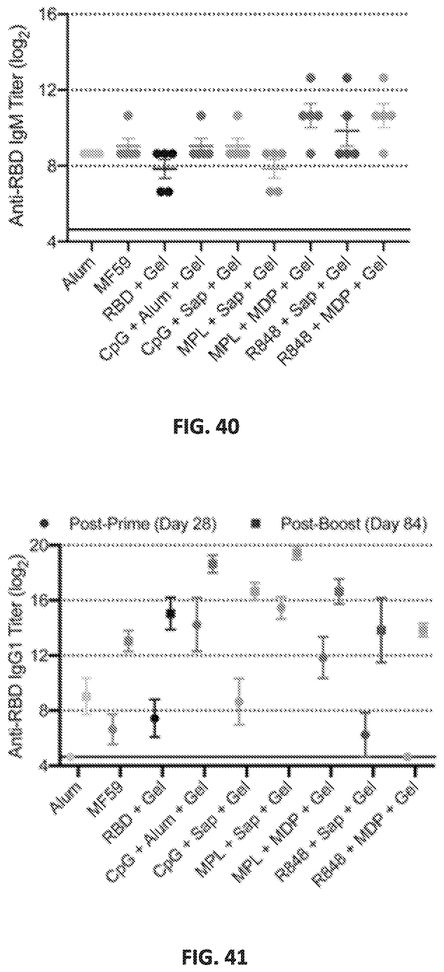

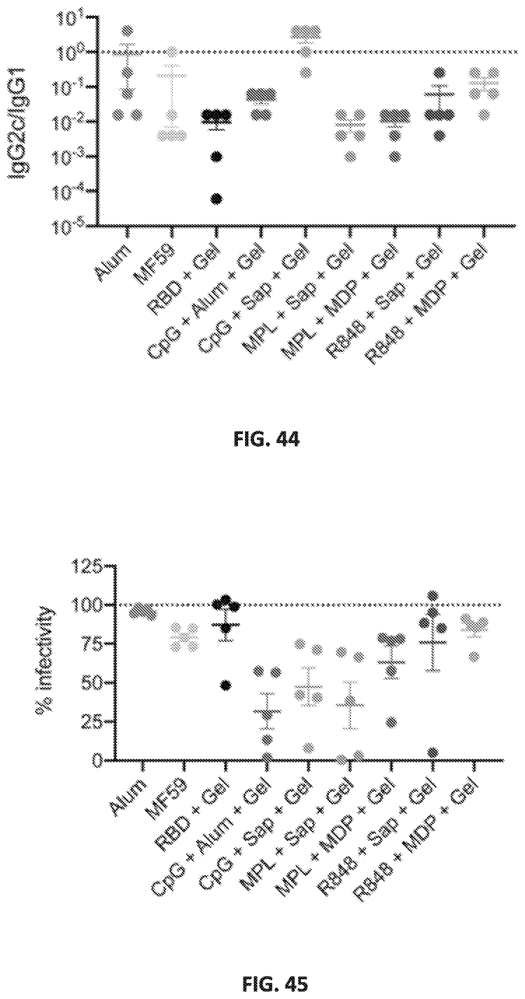

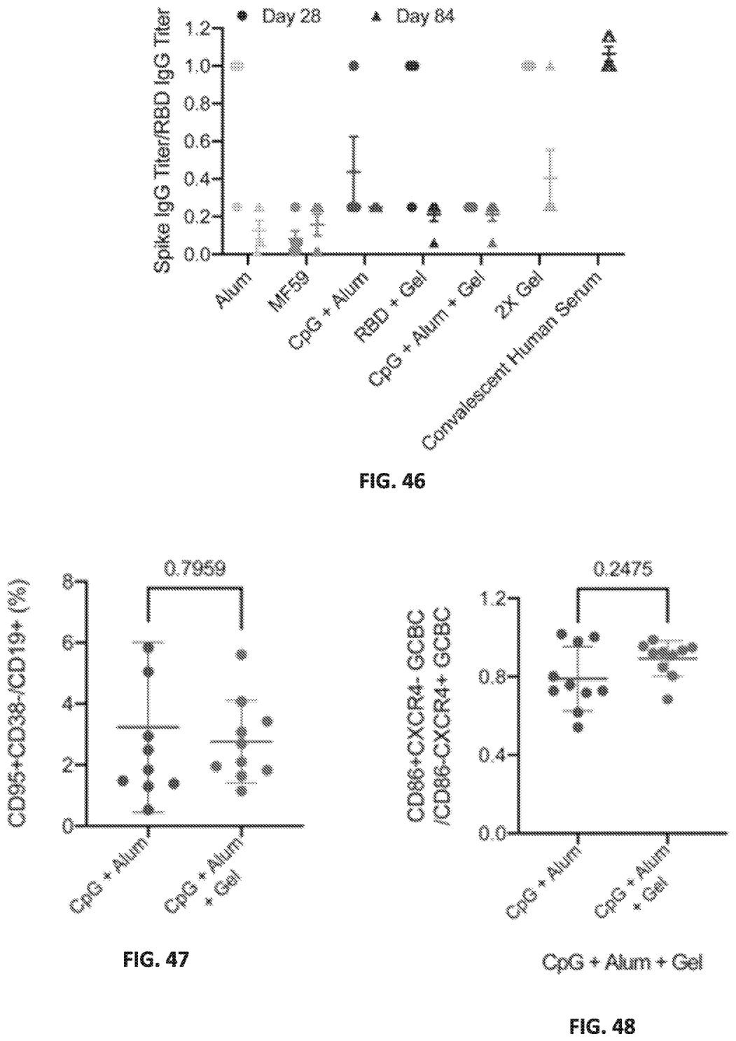

IgM is the first antibody isotype produced in response to vaccination prior to class switching. The function of IgM antibodies is to recognize and eliminate pathogens in the early stage of immune defense. On day 7 following immunization, we observed consistent IgM titers across groups (FIG. 40). Next, anti-RBD IgG1, IgG2b, and IgG2c titers were determined 4-weeks after both the prime and boost immunizations. RBD-specific IgG1 titers followed a similar trend to total IgG titers (FIGS. 16 and 41-43). The CpG+QuilA hydrogel (CpG+Sap+Gel) led to the highest IgG2b and IgG2c titers (FIGS. 41-43). CpG+Alum+Gel and CpG+Alum bolus treatments led to higher IgG2b titers than Alum and MF59 controls (FIG. 18). Although the CpG+Sap+Gel and CpG+Alum+Gel groups maintained high IgG2c titers, the clinically relevant controls (Alum and MF59) were much lower (FIGS. 18 and 41-43). The ratio of IgG2c to IgG1 titers is often used as a metric for Th1 versus Th2 skewing. We found that that the CpG+Alum+Gel treatment and RBD+Gel control led to the lowest ratio, suggesting a stronger Th2 or humoral response (FIG. 19). Alum, MF59, and the bolus control had ratios closer to 1 indicating a more balanced Th1/Th2 response (FIG. 19). With the exception of the CpG+Sap hydrogel group, hydrogel treatments tended to skew towards a stronger humoral response (FIG. 44). Since RBD is presented to the immune system as part of the spike protein, we also measured anti-spike IgG titers to assess the breadth of the response to the native spike protein. We observed a similar pattern to what was seen for anti-RBD IgG titers where the CpG+Alum+Gel treatment led to the highest titers both before and after boosting (FIGS. 20, 38, and 39). Overall, the anti-spike titers were slightly lower than anti-RBD titers as expected since we vaccinated with RBD. We also evaluated the ratio of anti-spike to anti-RBD IgG titers to better understand if the vaccines mostly produced antibodies against the RBD portion of spike or spike more broadly. The human convalescent serum (collected 9-10 weeks after symptom onset) was used as a positive control in this case since those patients were exposed to the complete virus and not just a specific RBD or spike protein. Across most groups, a fraction of the mice had a relatively high anti-spike to anti-RBD ratio at the early timepoint, but by the later timepoint the ratio dramatically declined and deviated from the ratio observed for convalescent human serum (FIG. 46).

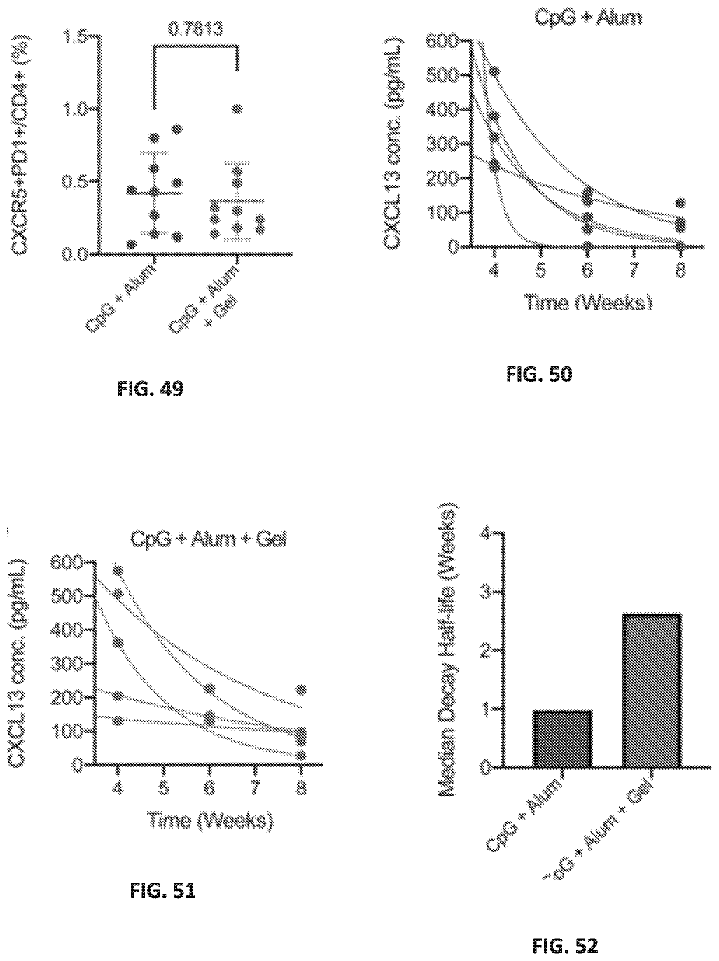

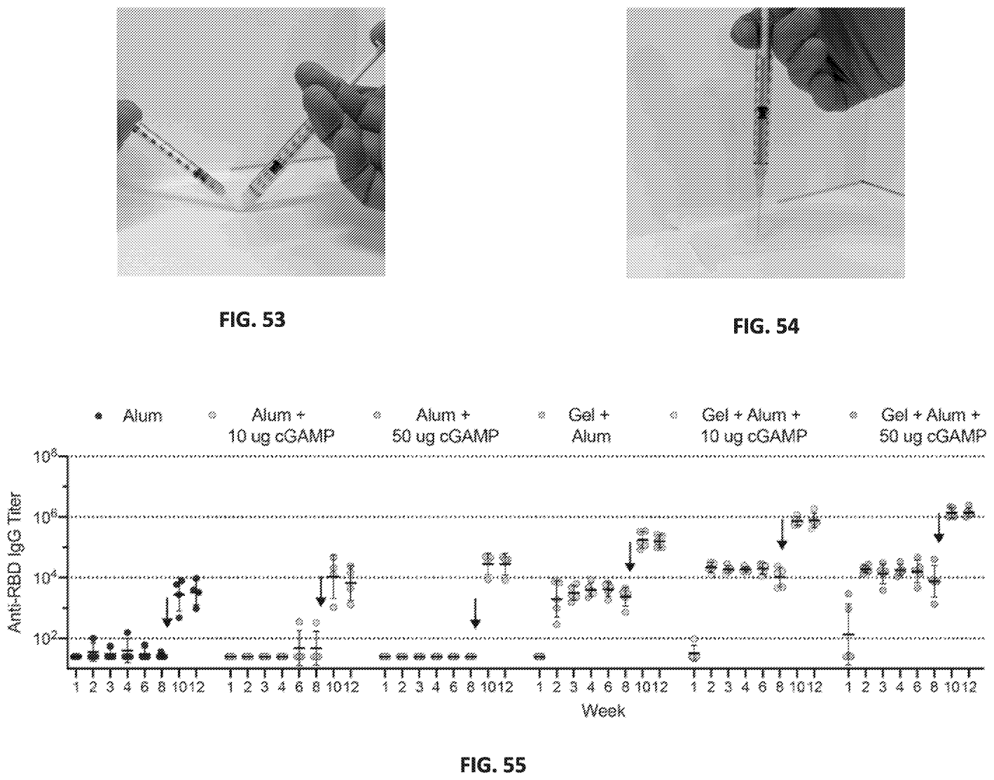

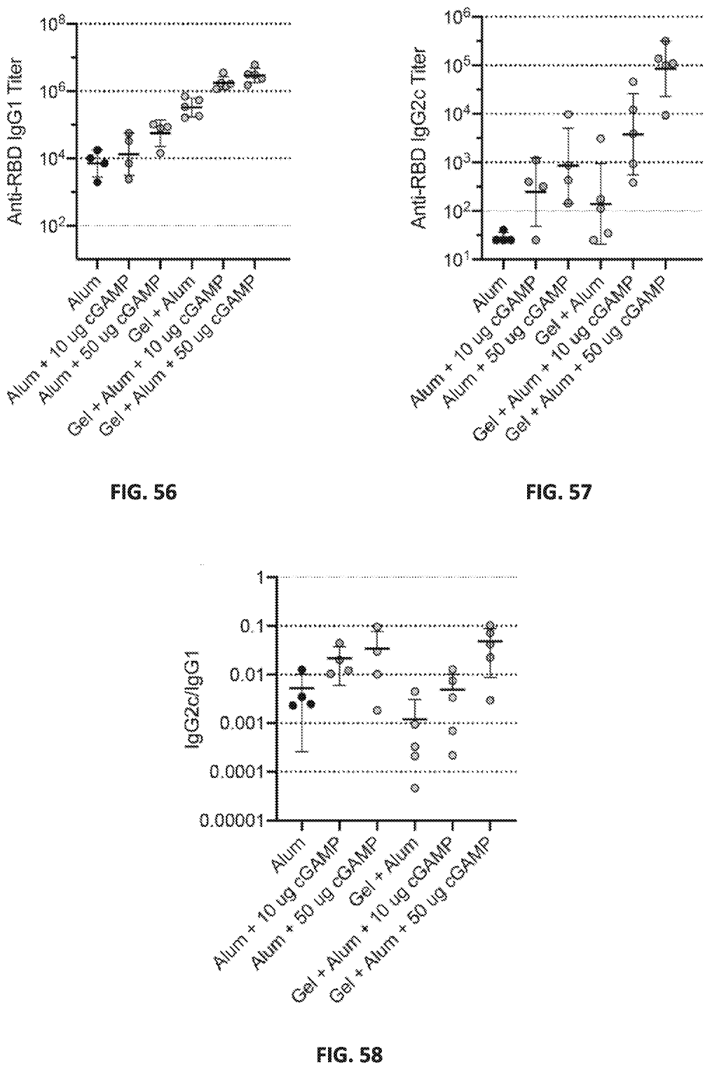

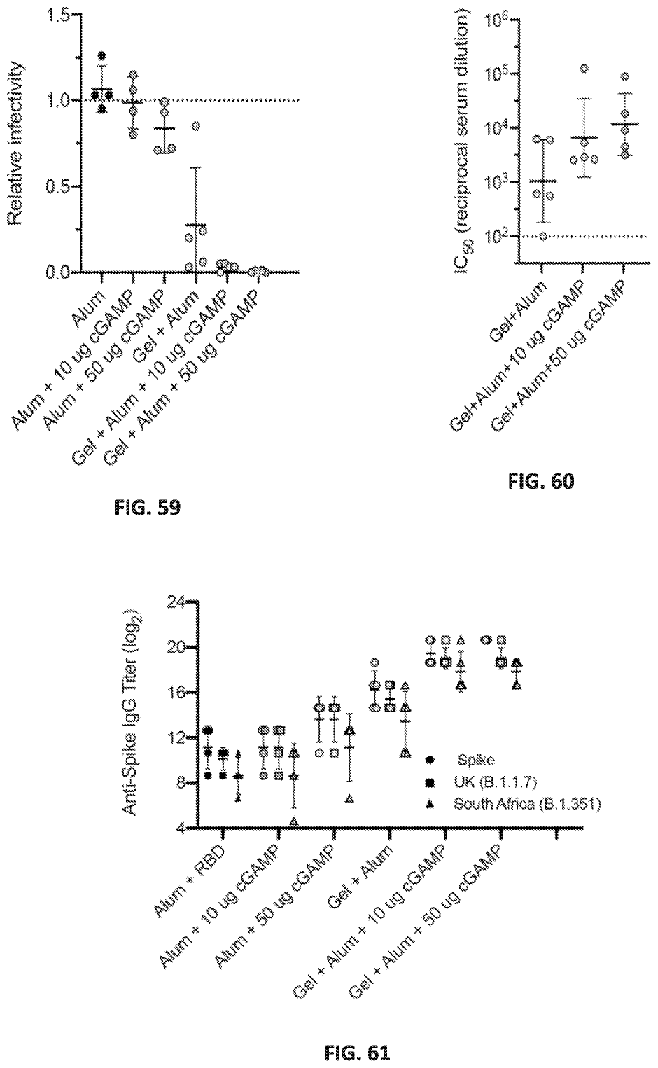

Previous work from our lab showed that prolonged germinal center (GC) activity following hydrogel vaccination led to a robust humoral response. We immunized mice with the CpG+Alum+Gel vaccine or the dose-matched bolus control and assessed GC activity at week 2 when the GC response to a bolus immunization should be strongest. There was no difference in frequency of GC B cells, light zone/dark zone ratio, or frequency of T follicular helper T cells at this time point (FIGS. 47-49). Next, we compared GC activity over longer time scales to determine if the hydrogel vaccine led to an extended response compared to the bolus vaccine. We measured the concentration of CXCL13 in serum since it is a biomarker of GC activity and could be quantified from an available serum from 4-8 weeks post-vaccination (FIGS. 50 and 51). The median half-life of CXCL13 decay from its peak at week 4 was extended from about 1 week after to over 2.5 weeks when the vaccine was delivered from the hydrogel (FIG. 52).

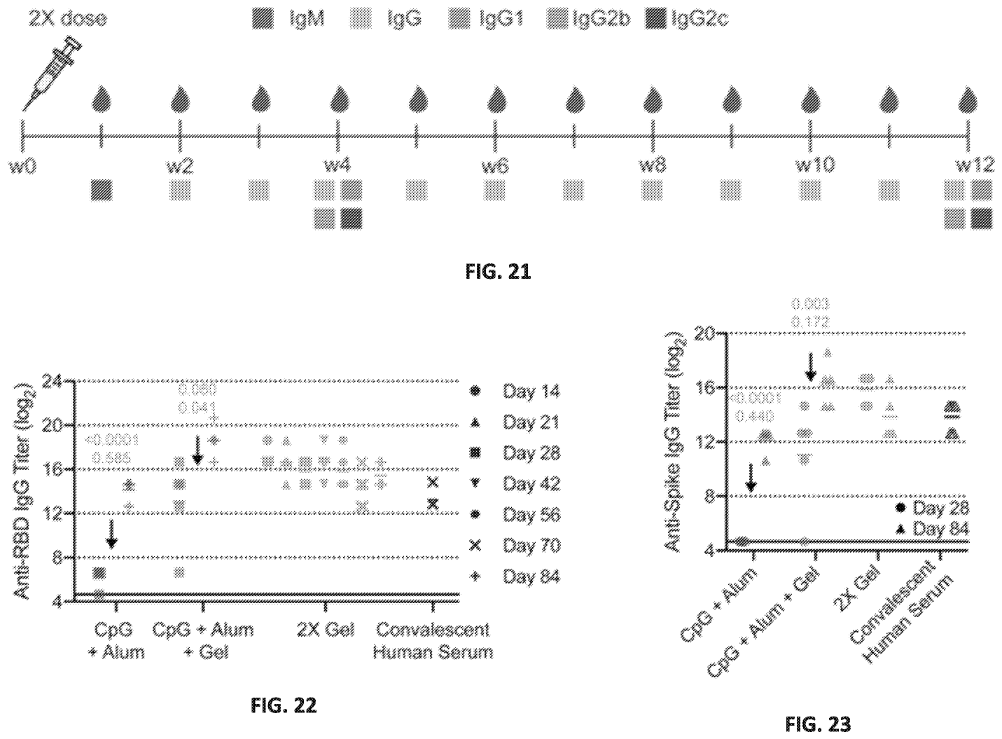

An ideal COVID-19 vaccine would require only a single immunization. We therefore designed a single-injection vaccine based on the CpG+Alum+Gel treatment that contained double the dose of all components with the goal of inducing similarly high titers and neutralization (FIG. 21). IgG titers from the single-injection vaccine (2× Gel) exceeded those of post-prime single dose gel titers and exceeded the post-boost titers of the bolus control (FIG. 22). Without boosting, the 2× Gel IgG titers persisted for 12 weeks. The anti-RBD IgG titers also remained above the titers of convalescent human serum showing that the vaccine efficacy in mice surpasses immunity following a natural infection in humans. Strikingly, the anti-spike IgG 2× Gel titers were almost equivalent to the post-boost CpG+Alum+Gel titers, suggesting a single shot achieved the same humoral response to the native spike protein (FIG. 23). The anti-spike titers for the 2× Gel also persisted and remained above the anti-spike titers for the bolus group through week 12 and remained at or above the titer levels of the convalescent human patient serum (FIG. 23).

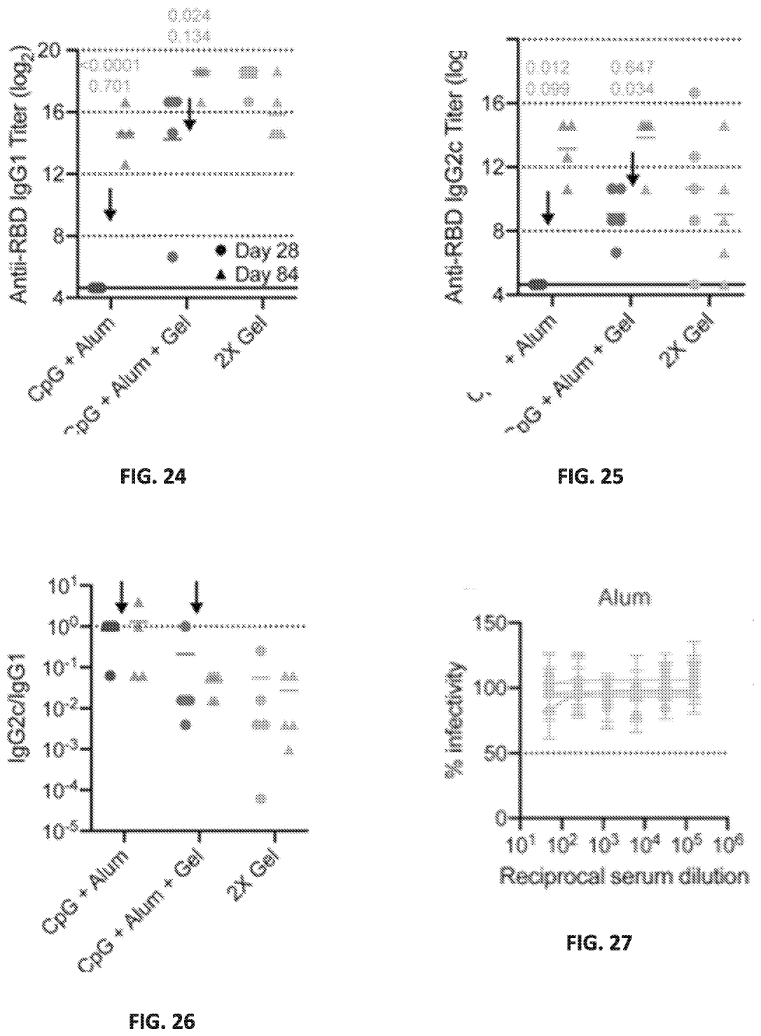

In order to further compare the response following 2× Gel treatment to the prime-boost responses, we also measured 2× Gel IgG1 and IgG2c titers. 2× Gel titers generally followed a similar trend to the CpG+Alum+Gel titers with higher mean IgG1 titers and lower mean IgG2c titers compared to the bolus (FIGS. 24 and 25). The IgG2c/IgG1 ratio was similar to that of the CpG+Alum+Gel group, suggesting a strong humoral response as expected (FIG. 26).

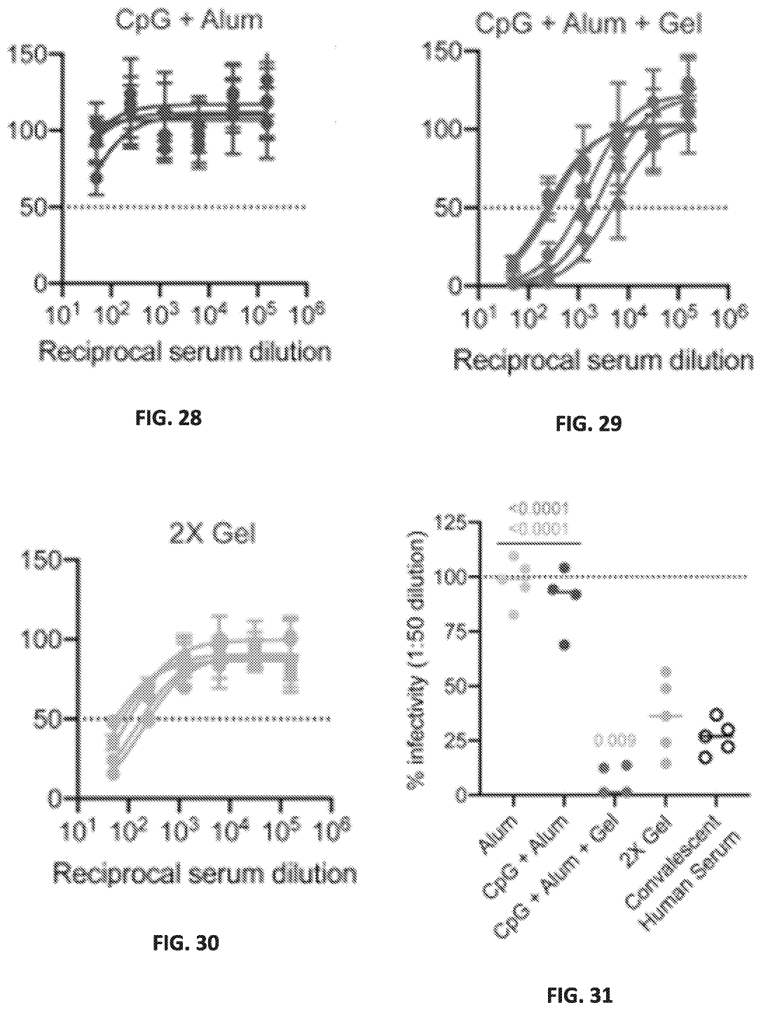

ELISA titers provide a useful measure for understanding antibody binding. In comparison, functional assays like neutralization assays with pseudotyped viruses provide additional information about the humoral response by quantifying antibody-mediated viral inhibition. Recent work found that neutralization ability as determined by a similar spike-pseudotyped neutralization assay correlated strongly with protection from a SARS-COV-2 challenge in non-human primates. To analyze neutralizing titers, we used lentivirus pseudotyped with SARS-COV-2 spike and assessed inhibition of viral entry into HeLa cells overexpressing human ACE2. We assessed the presence of neutralizing antibodies in serum collected 4 weeks after the final immunization (week 12 for all prime/boost groups and week 4 for the 2× Gel group). An initial screen with a 1:250 serum dilution showed that the average percent infectivity was reduced more than 50% for the CpG+Alum+Gel, CpG+Sap+Gel, and MPL+Sap+Gel treatments (FIG. 45). The control groups (Alum and RBD+Gel) showed less significant reductions in infectivity with the exception of MF59 which showed a slight decrease in average infectivity (˜20%). A series of serum dilutions was then run with experimental duplicates with serum from mice that received CpG+Alum treatments (either gel or bolus) or Alum alone (FIGS. 27-30). The CpG+Alum bolus treatment did not notably reduce infectivity even at high serum concentrations (FIGS. 27-30). In contrast, serum from mice that received a prime and boost of the CpG+Alum+Gel was completely neutralizing at high concentrations and the IC50 could be quantified from dose-inhibition curves from all samples (FIGS. 27-32). The 2× Gel group also resulted in neutralization in all mice, with all curves reaching the IC50 mark, but the curves were left-shifted compared to the curves from mice that received two immunizations.

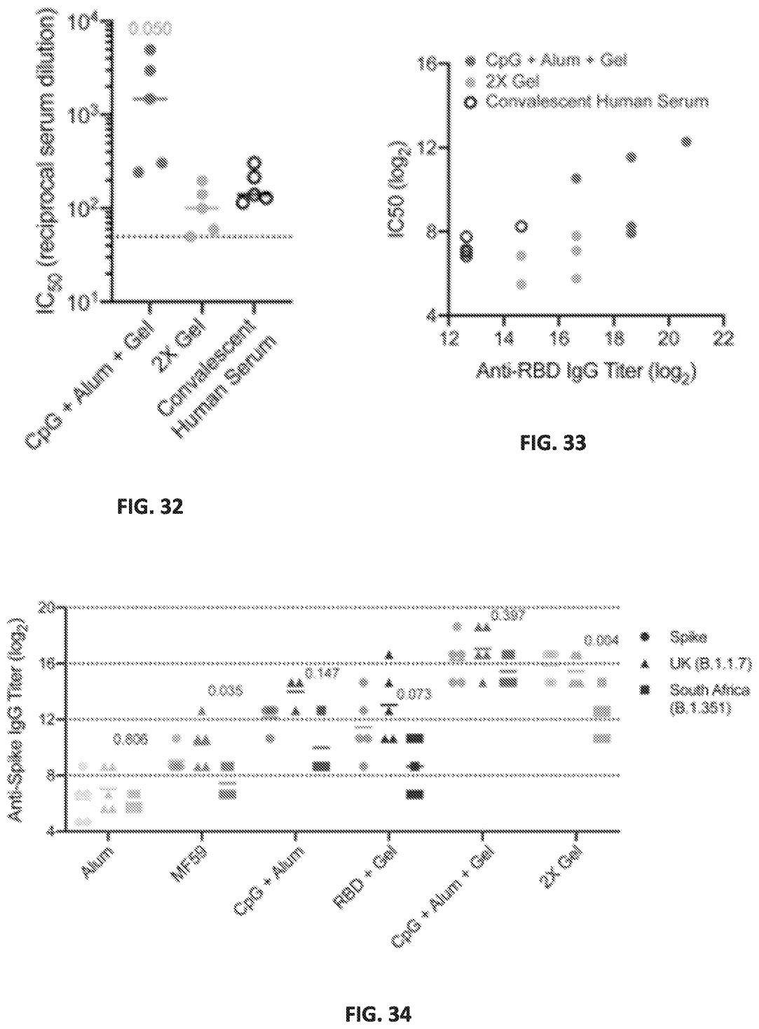

By plotting percent infectivity at a 1:50 serum dilution the differences between groups are distinct, with hydrogel treatments affording greater protection (FIG. 31). By this metric, antibodies from convalescent human serum provide similar protection to those from the hydrogel groups (FIG. 31). Overall, quantifiable levels of neutralizing antibodies were observed in all mice that received two immunizations of the 1×CpG+Alum+Gel and all mice that received a single 2× Gel immunization, although at lower levels (FIG. 32). No neutralization was detected in samples from mice that received either Alum or the CpG+Alum bolus treatment so IC50 values could not be determined (FIG. 32). Markedly, the CpG+Alum+Gel prime/boost treatment led to antibodies with a mean IC50 value that was about an order of magnitude greater than the mean IC50 value from antibodies in the convalescent human serum samples (FIG. 32). Since antibody titers are often used as a proxy for humoral response and the resulting protection, we plotted the relationships between anti-RBD IgG titer and neutralization IC50 values (FIG. 33). Although the correlation is quite weak (R2˜ 0.4), there is a positive correlation between the two metrics (FIG. 33).

Several different SARS-COV-2 variants that have increased transmission rates have been identified in countries including the UK, South Africa, and Brazil. Preliminary studies have been conducted to determine if previous infection and/or immunization with current vaccines protects against these variants. Although the Moderna and Pfizer/BioNTech vaccines are thought to provide robust protection against the UK variant, results vary against the South African variant. Recent research has found that cross-reactive neutralizing antibodies to RBD generally do not overlap with sites on RBD that have mutated during antigenic drift, signifying RBD might be a useful target antigen for achieving a broad response. SARS-COV-2 can be expected to continue to mutate until protective immunizations are distributed equitably around the globe. We therefore assessed titers against the UK and South Africa spike variants following immunization with the vaccines disclosed herein to assess if broad protection resulted. All vaccines led to similar titers against the UK mutant and the wildtype form, but titers against the South Africa variant were noticeably lower compared to the wildtype form across all groups except Alum and CpG+Alum+Gel (FIG. 34). Although titers against the South Africa variant were lower for the 2× Gel group compared to wildtype spike, they still matched the post-boost CpG+Alum bolus titers for wildtype spike, suggesting that one injection of the 2× Gel afforded a similar level of protection against all variants as two injections of a standard CpG+Alum bolus (FIG. 34).

The innate second messenger 2′3′-cyclic-GMP-AMP (cGAMP) is on the forefront of adjuvant design to elicit anti-viral and anti-cancer immunity through its activation of Stimulator of Interferon Genes (STING) signaling. In the quest to rapidly develop an efficacious SARS-COV-2 vaccine, the virus's spike (S) protein, specifically its receptor binding domain (RBD), has emerged as a promising target for the generation of neutralizing antibodies. However, the success of subunit vaccine strategies is often hampered by poor inherent immunogenicity. We seek to answer the following question to further the development of a SARS-COV-2 subunit vaccine: is cGAMP a more potent adjuvant than first-generation innate immune adjuvants (e.g. TLR agonists). We hypothesize that co-administration of cGAMP with RBD is a promising strategy to stimulate acute antiviral inflammatory cytokine signaling and boost generation of high-affinity neutralizing antibodies against SARS-COV-2.

Activation of the cGAMP-STING pathway unleashes a powerful anti-viral innate immune program. Upon detection of mis-localized double-stranded DNA in the cytosol, such as from viral infection or cell damage, cyclic-GMP-AMP-synthase (cGAS) catalyzes production of cGAMP from ATP and GTP. Canonically, cGAMP serves as an intracellular second messenger: cGAMP binding activates STING, triggers activation of the kinase TBK1 and transcription factor IRF3, and results in production of type I interferons and other anti-viral cytokines. Remarkably, cGAMP also acts as an extracellular messenger to rapidly signal danger across the local environment. Of note, cGAMP signaling acts by being exported and imported by cells via specific mechanisms. cGAMP is of interest as it is a small molecule with drug-like properties. While TLR-targeting adjuvants also result in potent type I interferon production, delivery of large, polymeric ligands (e.g., dsRNA/poly(I:C)) to cells is challenging and typically requires encapsulation strategies to increase its cellular uptake and downstream signaling.

In a recent report, delivery of cGAMP-loaded pulmonary surfactant biomimetic nanoparticles with non-replicating intranasal influenza vaccines resulted in heterosubtypic anti-influenza immunity. Intratumoral administration of the non-hydrolyzable cGAMP analog ADU-S100 is being evaluated in clinical trials (NCT03172936, NCT03937141) for its ability to boost tumor immunogenicity and synergize with checkpoint inhibitors. cGAMP and its analogs are of interest due to the urgent need to develop a SARS-COV-2 vaccine.

Materials. HPMC (meets USP testing specifications), N,Ndiisopropylethylamine (Hunig's base), hexanes, diethyl ether, N-methyl-2-pyrrolidone (NMP), dichloromethane (DCM), lactide (LA), 1-dodecylisocynate, and diazobicylcoundecene (DBU) were purchased from Sigma-Aldrich and used as received. Monomethoxy-PEG (5 kDa) was purchased from Sigma-Aldrich and was purified by azeotropic distillation with toluene prior to use.

Preparation of HPMC-C12. HPMC-C12 was prepared according to previously reported procedures. HPMC (1.0 g) was dissolved in NMP (40 mL) by stirring at 80° C. for 1 hr. Once the solution reached room temperature (RT), 1-dodecylisocynate (105 mg, 0.5 mmol) and N,N-diisopropylethylamine (catalyst, ˜3 drops) were dissolved in NMP (5.0 mL). This solution was added dropwise to the reaction mixture, which was then stirred at RT for 16 hr. This solution was then precipitated from acetone, decanted, redissolved in water (˜2 wt %), and placed in a dialysis tube for dialysis for 3-4 days. The polymer was lyophilized and reconstituted to a 60 mg/mL solution with sterile PBS.

Preparation of PEG-PLA NPs. PEG-PLA was prepared as previously reported. Monomethoxy-PEG (5 kDa; 0.25 g, 4.1 mmol) and DBU (15 μL, 0.1 mmol; 1.4 mol % relative to LA) were dissolved in anhydrous dichloromethane (1.0 mL). LA (1.0 g, 6.9 mmol) was dissolved in anhydrous DCM (3.0 mL) with mild heating. The LA solution was added rapidly to the PEG/DBU solution and was allowed to stir for 10 min. The reaction mixture was quenched and precipitated by a 1:1 hexane and ethyl ether solution. The synthesized PEG-PLA was collected and dried under vacuum. Gel permeation chromatography (GPC) was used to verify that the molecular weight and dispersity of polymers meet our quality control (QC) parameters. NPs were prepared as previously reported. A 1-mL solution of PEG-PLA in DMSO (50 mg/mL) was added dropwise to 10 mL of water at RT under a high stir rate (600 rpm). NPs were purified by centrifugation over a filter (molecular weight cutoff of 10 kDa; Millipore Amicon Ultra-15) followed by resuspension in PBS to a final concentration of 200 mg/mL. NPs were characterized by dynamic light scattering (DLS) to find the NP diameter, 37±4 nm.

PNP Hydrogel Preparation. The hydrogel formulation contained 2 wt % HPMC-C12 and 10 wt % PEG PLA NPs in PBS. These gels were made by mixing a 2:3:1 weight ratio of 6 wt % HPMC-C12 polymer solution, 20 wt % NP solution, and PBS containing all other vaccine components. The NP and aqueous components were loaded into one syringe, the HPMC-C12 was loaded into a second syringe and components were mixed using an elbow connector. After mixing, the elbow was replaced with a 21-gauge needle for injection.

Material Characterization. Rheological characterization was performed on PNP hydrogels with or without Alum (Alhydrogel) using a TA Instruments Discovery HR-2 torque-controlled rheometer (TA Instruments) fitted with a Peltier stage. All measurements were performed using a serrated 20-mm plate geometry at 25° C. with a 700 μm gap height. Dynamic oscillatory frequency sweep measurements were performed from 0.1 to 100 rad/s with a constant oscillation strain in the linear viscoelastic regime (1%). Amplitude sweeps were performed at a constant angular frequency of 10 rad/s from 0.01% to 10,000% strain with a gap height of 500 μm. Steady shear experiments were performed by alternating between a low shear rate (0.1 s⁻¹) and high shear rate (10 s⁻¹) for 60 seconds each for three full cycles. Shear rate sweep experiments were performed from 10 s⁻¹ to 0.001 s⁻¹.

Expression and Purification of RBD. The mammalian expression plasmid for RBD production was previously described in detail in (Amanat et al., 2020, Nat Medicine). RBD was expressed and purified from Expi293F cells as previously described. Briefly, Expi293F cells were cultured using 66% FreeStyle293 Expression/33% Expi293 Expression medium (Thermo Fisher) and grown in polycarbonate baffled shaking flasks at 37° C. and 8% CO2 while shaking. Cells were transfected at a density of approximately 3-4×10⁶ cells/mL. Cells were harvested 3-5 days post-transfection via centrifugation. RBD was purified with HisPur NiNTA resin (Thermo Fisher). Resin/supernatant mixtures were added to glass chromatography columns for gravity flow purification. Resin was washed with 10 mM imidazole/1×PBS [pH 7.4] and proteins were eluted. NiNTA elutions were concentrated using Amicon spin concentrators (10-kDa MWCO for RBD) followed by size-exclusion chromatography. The RBD was purified using a GE Superdex 200 Increase 10/300 GL column. Fractions were pooled based on A280 signals and/or SDS-PAGE. Samples for immunizations were supplemented with 10% glycerol, filtered through a 0.22-μm filter, snap frozen, and stored at −20° C. until use.

Vaccine Formulations. The vaccines contained a 10-μg dose of RBD and combinations of 5 μg Quil-A Adjuvant (Invivogen), 50 μg Resiquimod (R848; Selleck Chemicals), 20 μg L18-MDP (Invivogen), 10 μg MPLA (Invivogen), 20 μg CpG ODN 1826 (Invivogen), or 100 μg Alhydrogel in 100 μL hydrogel or PBS based on the treatment group. For the bolus vaccines, the above vaccine doses were prepared in PBS and loaded into a syringe for administration. For the PNP hydrogels, the vaccine cargo was added at the appropriate concentration into the PBS component of the gel and combined with the NP solution before mixing with the HPMC-C12 polymer, as described above.

RBD and CpG Gel Release Studies. Hydrogels were prepared the same way as described in the “PNP Hydrogel Preparation” section and were loaded with 10 μg RBD, 20 μg CpG and 100 μg Alum. Glass capillary tubes were plugged at one end with epoxy and 100 μL of gel was injected into the bottom of 3 different tubes. 350 μL of PBS was then added on top of each gel. The tubes were stored upright in an incubator at 37° C. for about 3 weeks. At each timepoint, ˜300 μL of PBS was removed and the same amount was replaced. The amount of RBD released at each timepoint was determined using a Micro BCA™ Protein Assay Kit (Fisher Scientific) following the manufacturer's instructions (including using the Bovine Serum Albumin standards provided in the kit). The amount of CpG released was determined by measuring the absorbance at 260, subtracting the absorbance from a blank well with buffer, and then applying the Beer-Lambert law with an extinction coefficient of 0.027 μg/mL*cm-1 for single-stranded DNA. For both types of cargo, the cumulative release was calculated and normalized to the total amount released over the duration of the experiment. For CpG retention, the points were fit with a one-phase decay in GraphPad Prism and the half-life of release was determined (n=3). For RBD retention, the points were fit with a linear fit in GraphPad Prism and the half-life of release was determined (n=3).

Alexa Fluor 647-conjugated RBD was synthesized by the following methods: AFDye 647-NHS ester (Click Chemistry Tools, 1.8 mg, 1.85 μmol) was added to a solution of RBD protein (0.84 mg, 0.926 μmol) in PBS. The NHS ester reaction was conducted with a 20 molar excess of AFDye 647-NHS ester to RBD in the dark for 3 hr at RT with mild shaking. The solution was quenched by diluting 10-fold with PBS. The solution was then purified in centrifugal filters (Amicon Ultra, MWCO 10 kDa) at 4500 RCF for 20 min, and the purification step was repeated until all excess dye was removed.

Vaccine-loaded hydrogels or a bolus controls with 10 μg Alexa Fluor 647-conjugated RBD, 20 μg CpG and 100 μg Alum were injected into mice and fluorescence was monitored over time by In Vivo Imaging System (IVIS Lumina Imager; Ex=600 nm, Em=670 nm). Images were collected on days 1, 4, 7, 11, 13, and 18 (n=5 mice). Signal was quantified as raw fluorescence within a constant region of interest. GraphPad Prism was used to fit one-phase decays with a constrained initial value based on day 0 signal and half-lives of release were determined (n=5).

Mice and Vaccination. C57BL/6 mice were purchased from Charles River and housed at Stanford University. 8-10 week-old female mice were used. Mice were shaved prior to initial immunization. Mice received 100 μL hydrogel or bolus vaccine on their backs under brief isoflurane anesthesia. Bolus treatments were injected with a 26-gauge needle and hydrogels were injected with a 21-gauge needle. Mouse blood was collected from the tail vein for survival bleeds over the course of the study.

Mouse Serum ELISAs. Anti-RBD and Anti-spike trimer antibody titers were measured using an end-point ELISA. 96-well Maxisorp plates (Thermo Fisher) were coated with RBD, full-length spike35, the mutant spike from the UK B.1.1.7 (Sino Biological 40591-V08H12), or the mutant spike from South Africa B.1.351 (Sino Biological 40591-V08H10) at 2 μg/mL in 1×PBS [pH 7.4] overnight at 4° C. Plates were then blocked with 1% bovine serum albumin (BSA in 1×PBS) for 1 hr at RT. Serum samples were serially diluted starting at a 1:100 dilution and incubated on blocked plates for 2 hr at RT. One of the following goat-anti-mouse secondary antibodies was used: IgG Fc-HRP (1:10,000, Invitrogen A16084), IgG1 heavy chain HRP (1:50,000, abcam ab97240), IgG2b heavy chain HRP (1:10,000, abcam ab97250), IgG2c heavy chain HRP (1:10,000, abcam ab97255), or IgM mu chain HRP (1:10,000 abcam ab97230). The secondary antibody was added at the dilution listed (in 1% BSA) for 1 hr at RT. 5×PBS-T washes were done between each incubation step. Plates were developed with TMB substrate (TMB ELISA Substrate (High Sensitivity), Abcam). The reaction was stopped with 1 M HCl. Plates were analyzed using a Synergy H1 Microplate Reader (BioTek Instruments) at 450 nm. End-point titers were defined as the highest serum dilution that gave an optical density above 0.1. For plots displaying a single time point, P values listed were determined using a one-way ANOVA and for plots displaying multiple timepoints, P values listed were determined using a 2-way ANOVA. Both statistical analyses were done using Tukey's multiple comparisons test on GraphPad Prism software. All titer data is shown as the mean and individual points (n=5) with P values listed above the points.

Mouse IFNα All Subtype ELISA kit, High Sensitivity (PBL Assay Science, 42115-1), Mouse TNFα Quantikine ELISA kit (R&D Systems, SMTAOOB), and Legend Max Mouse CXCL13 (BLC) ELISA kit (BioLegend, 441907) were used to quantify different serum cytokines. Serum dilutions of 1:10 were used for all ELISAs. Concentrations were determined by ELISA according to manufacturer's instructions. Absorbance was measured at 450 nm in a Synergy H1 Microplate Reader (BioTek). Cytokine concentrations were calculated from the standard curves which were run in technical duplicate. Concentration data are reported as ng/mL for IFNα and TNFα and pg/mL for CXCL13 and displayed as individual points and the mean.

Immunophenotyping in Lymph Nodes. Methods from previous germinal center phenotyping done in the lab were followed. Briefly, inguinal lymph nodes were removed from mice after euthanasia and were disrupted to create a cell suspension. For flow cytometry analysis, cells were blocked with anti-CD16/CD38 (clone: 2.4G2) and then stained with fluorochrome-conjugated antibodies: CD19, GL7, CD95, CXCR4, CD86, IgG1, CD4, CXCR5, and PD1. Cells were then washed, fixed, and analyzed on an LSRII flow cytometer. Data were analyzed with FlowJo 10 (FlowJo LLC).

SARS-COV-2 spike-pseudotyped Viral Neutralization Assay. Neutralization assays were conducted as described previously. Briefly, SARS-COV-2 spike-pseudotyped lentivirus was produced in HEK239T cells. Six million cells were seeded one day prior to transfection. A five-plasmid system was used for viral production. Plasmids were added to filter-sterilized water and HEPES-buffered saline was added dropwise to a final volume of 1 mL. CaCl₂) was added dropwise while the solution was agitated to form transfection complexes. Transfection reactions were incubated for 20 min at RT, then added to plated cells. Virus-containing culture supernatants were harvested ˜72 hours after transfection by centrifugation and filtered through a 0.45-μm syringe filter. Stocks were stored at −80° C.

For the neutralization assay, ACE2/HeLa cells were plated 1-2 days prior to infection. Mouse serum was heat inactivated at 56° C. for 30 min prior to use. Mouse serum and virus were diluted in cell culture medium and supplemented with a polybrene at a final concentration of 5 μg/mL. Serum/virus dilutions were incubated at 37° C. for 1 hr. After incubation, media was removed from cells and replaced with serum/virus dilutions and incubated at 37° C. for 2 days. Cells were then lysed using BriteLite (Perkin Elmer) luciferase readout reagent, and luminescence was measured with a BioTek plate reader. Each plate was normalized by wells with cells only or virus only and curves were fit with a three-parameter non-linear regression inhibitor curve to obtain IC50 values. Serum samples that failed to neutralize or that neutralized at levels higher than 1:50 were set at the limit of quantitation for analyses. Serum dilution curves display mean infectivity +/−SEM for each individual mouse (n=5) at each serum dilution. Normalized values were fit with a three-parameter non-linear regression inhibitor curve in GraphPad Prism to obtain IC50 values. Fits were constrained to have a value of 0% at the bottom of the fit. Single dilution infectivity plots and IC50 data are shown as individual mouse or human titer values (n=5) and the mean. P values listed were determined in GraphPad Prism software using a one-way ANOVA with Tukey's multiple comparison test.

Collection of Serum from Human Patients. Convalescent COVID-19 blood was collected from 5 donors 9-10 weeks after onset of symptoms. Blood was collected in microtubes with serum gel for clotting (Starstedt), centrifuged for 5 minutes at 10,000 g and then serum was stored at −80° C. until used. Blood collection was done by finger-prick.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected,” “attached,” or “coupled” to another feature or element, it can be directly connected, attached or coupled to the other feature or element or intervening features or elements may be present. In contrast, when a feature or element is referred to as being “directly connected,” “directly attached,” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A vaccine delivery system, comprising:
a hydrogel comprising a polymer non-covalently crossed-linked with a plurality of nanoparticles;
a dinucleotide adjuvant encapsulated in the hydrogel; and
an antigen encapsulated in the hydrogel.

2. The vaccine delivery system of claim 1, wherein the dinucleotide adjuvant comprises CpG.

3. The vaccine delivery system of claim 1, wherein the dinucleotide adjuvant comprises a cyclic dinucleotide.

4. The vaccine delivery system of claim 3, wherein the cyclic dinucleotide comprises cGAMP.

5. The vaccine delivery system of any one of claims 1-4, wherein the antigen comprises a receptor binding domain (RBD) of a virus.

6. The vaccine delivery system of claim 5, wherein the virus is a SARS-COV virus, a SARS-COV-2 virus, or a MERS-COV virus.

7. The vaccine delivery system of any of claims 1-6, wherein the polymer comprises hydroxypropylmethylcellulose (HPMC), or a derivative thereof.

8. The vaccine delivery system of any one of claims 1-7, wherein the nanoparticles are polymeric nanoparticles.

9. The vaccine delivery system of claim 8, wherein the polymeric nanoparticles comprise poly(ethylene glycol)-bpoly(lactic acid) (PEG-PLA).

10. The vaccine delivery system of any one of claims 1-9, further comprising:
an aluminum or aluminum salt adjuvant encapsulated in the hydrogel.

11. The vaccine delivery system of claim 10, wherein the aluminum or aluminum salt adjuvant comprises aluminum hydroxide.

12. The vaccine delivery system of any one of claims 1-11, further comprising:
one or more additional adjuvants selected from the list consisting of Resiquimod (R848), Monophosphoryl lipid A (MPL), Quil-A (Sap), and the fatty-acid modified form of muramyl dipeptide (MDP).

13. A method for inducing an immune response against the antigen of the vaccine delivery system of any one of claims 1-12 in a subject, the method comprising:
administering to the subject a therapeutically effective amount of the vaccine delivery system.

14. The method of claim 13, wherein the immune response comprises increased production of IgG antibodies.

15. The method of claim 14, wherein the immune response comprises increased production of IgG1 antibodies.

16. The method of claim 14 or 15, wherein the immune response comprises increased production of IgG2b antibodies.

17. The method of any one of claims 14-16, wherein the immune response comprises increased production of IgG2c antibodies.

18. The method of any one of claims 14-17, wherein the ratio of the concentration of IgG2c to the concentration of IgG1 in a serum sample from the subject taken after the administering is less than 0.3:1.

19. A method of preventing or treating a disease in a subject, the method comprising:
administering to the subject a therapeutically effective amount of the vaccine delivery system of any one of claims 1-12.

20. The method of claim 19, wherein, subsequent to the administering, the dinucleotide adjuvant and the antigen release from the hydrogel into the subject at substantially the same rate.

21. The method of claim 19 or 20, wherein the disease is COVID-19.

22. The method of any one of claims 19-21, wherein administering the vaccine delivery system comprises injecting the vaccine delivery system into the subject.

23. A method for delivering a vaccine to a subject, the method comprising:
mixing a first solution comprising HPMC-C₁₂ in a first receptacle with a second solution comprising PEG-PLA, an antigen, and a dinucleotide adjuvant in a second receptacle, to thereby form a homogenous solid-like hydrogel;
shearing the hydrogel through a syringe to form a shear-thinned gel; and
delivering the hydrogel into an interior of the subject and forming a solid-like gel antigen and nucleotide adjuvant depot.

24. The method of claim 23, wherein at least the first receptacle or the second receptacle comprises the syringe.

25. The method of claim 23 or 24, wherein the solid-like gel antigen and dinucleotide adjuvant depot is configured to release antigen and dinucleotide in the subject for at least two weeks.

26. The method of any of claims 23-25, wherein the second solution further comprises one or more additional adjuvants selected from the list consisting of an aluminum or aluminum salt, Resiquimod (R848), Monophosphoryl lipid A (MPL), Quil-A (Sap), and the fatty-acid modified form of muramyl dipeptide (MDP).

27. A pharmaceutical agent kit comprising:
a first receptacle comprising a polymer;
a second receptacle comprising a nanoparticle, a dinucleotide adjuvant, and an antigen;
a connector piece configured to fluidically connect the first receptacle with the second receptacle; and
an instructional material.

28. The pharmaceutical agent kit of claim 27 wherein the polymer comprises dodecyl-modified hydroxypropylmethylcellulose (HPMC-C₁₂).

29. The pharmaceutical agent kit of claim 27 or 28, wherein the nanoparticle comprises poly(ethylene glycol)-bpoly(lactic acid) (PEG-PLA).

30. The pharmaceutical agent kit of any one of claims 27-29, wherein the first receptacle and the second receptacle comprise syringes.

31. The pharmaceutical agent kit of any one of claims 27-30, wherein the second receptacle comprises a receptor binding domain (RBD) of a virus.
